SEQUENCE LISTING (17 sequences)

SEQ ID NO: 1 - DNA, 669 bp, oryza sativa
atggcgcgaggcaaggtgcagctccgtcgcatcgagaacccggttcaccgtcaggtcaccttctgcaagcgccgtgccggcctgctgaagaaggccagggagctctccatcctctgcgaggccgacatcggcatcatcatcttctccgcccacggcaagctctacgacctcgccaccaccggaaccatggaggagctgatcgagaggtacaagagtgctagtggcgaacaggccaacgcctgcggcgaccagagaatggacccaaaacaggaggcaatggtgctcaaacaagaaatcaatctactgcagaagggcctgaggtacatctatgggaacagggcaaatgaacacatgactgttgaagagctgaatgccctagagaggtacttagagatatggatgtacaacattcgctccgcaaagatgcagataatgatccaagagatccaagcactaaagagcaaggaaggcatgttgaaagctgctaacgaaattctccaagaaaagatagtagaacagaatggtctgatcgacgtaggcatgatggtagcagatcaacagaatgggcattttagtacagtcccactgttagaagagatcactaacccactgactatactgagtggctattctacttgtaggggctcggagatgggctattccttctaa

SEQ ID NO: 2 - protein, 280 aa, oryza sativa
MARGKVQLRRIENPVHRQVTFCKRRAGLLKKARELSILCEADIGIIIFSAHGKLYDLATTGTMEELIERYKSASGEQANACGDQRMDPKQEAMVLKQEINLLQKGLRYIYGNRANEHMGTMEELIERYKSASGEQANACGDQRMDPKQEAMVLKQEINLLQKGLRYIYGNRANEHMTVEELNALERYLEIWMYNIRSAKMQIMIQEIQALKSKEGMLKAANEILQEKIVEQNGLIDVGMMVADQQNGHFSTVPLLEEITNPLTILSGYSTCRGSEMGYSF

SEQ ID NO: 3 - protein, 224 aa, triticum aestivum
MARGKVQLRRIENPVHRQVTFCKRRAGLLKKARELSVLCDADIGIIIFSAHGKLYDLATTGTMDGLIERYKSASGEGMTGDGCGDQRVDPKQEAMVLKQEIDLLQKGLRYIYGNRANEHMNVDELNALERYLEIWMFNIRSAKMQIMIQEIQALKSKEGMLKAANEILQEKIVEQHGLIDVGMTIADQQNGHFSTVPMLEEITNPLTILSGYSTCRGSEMGYSF

SEQ ID NO: 4 - DNA, 675 bp, triticum aestivum
atggcgagaggcaaggtccagctccggcgcatcgagaaccccgtccaccggcaggtcaccttctgcaagcgccgcgcagggctcctcaagaaggccagggagctctctgtcctctgcgacgccgacatcggcatcatcatcttctccgcacacggcaagctctacgacctcgccaccaccggaaccatggatgggctgatcgagaggtacaagagtgccagtggagaaggcatgaccggcgacggctgcggcgaccagagagtggacccaaagcaggaggcaatggtgctgaaacaagaaatagaccttctgcagaagggactgaggtacatttatggaaacagggcaaatgagcacatgaatgttgacgagctgaatgccctggagaggtacttggagatatggatgttcaacatccgctccgcaaagatgcagataatgattcaagagatccaggcactgaagagcaaggagggcatgttgaaagctgccaacgaaattctccaggaaaagatagtagaacagcatggactgatcgacgtaggcatgactatagcagatcagcagaatgggcattttagtacagtcccaatgttagaggagatcactaacccactgactatactgagtggctattctacttgtaggggctcagagatgggctattccttctga

SEQ ID NO: 5 - protein, 222 aa, sorghum bicolor
MARGKVQLRRIENPVHRQVTFCKRRAGLLKKARELSVLCDAHIGIIIFSAHGKLYDLATTGTMEELIDRYKTASGEAADGSGDNRMDPKQETMVLQQEINLLQKGLRYIYGNRANEHMNVDELNALERYLEIWMYNIRSAKMQIMIQEIQALKSKEGMLKAANEILREKIVEQSSLLDVGMVVADQQNGHFSTVPLIEEITNPLTILSGYSNCRGSEMGYSF

SEQ ID NO: 6 - DNA, 669 bp, sorghum bicolor
atggcgcggggcaaagtgcagctgcggcgcatcgagaacccggtgcaccggcaggtgaccttctgcaagcgccgcgcggggctgctcaagaaggcacgggagctctccgtcctctgcgacgcccacatcggcatcatcatcttctccgcgcacggcaagctctacgacctcgccaccaccgggaccatggaagagctgatcgacaggtacaagactgccagcggagaagctgccgacggctccggcgacaacagaatggatccaaaacaagaaaccatggtgctgcaacaggaaatcaatctgctccagaaaggactcaggtacatctacgggaacagggcaaatgaacacatgaatgttgacgaactgaatgcccttgagaggtacttggagatatggatgtacaacatccgctctgcaaagatgcagataatgattcaagagatacaagcactaaaaagcaaggaaggcatgttgaaagctgctaacgaaattctccgggaaaagatagtagaacagagtagtttgcttgatgtaggcatggtggtagcggatcaacagaatgggcattttagtacagtcccactgatagaagagatcactaacccactgactatactgagtggatattctaactgtaggggctcagagatgggctattccttctaa

SEQ ID NO: 7 - protein, 245 aa, zea mays
MGRGKVQLKRIENKINRQVTFSKRRSGLLKKAHEISVLCDAEVALIIFSTKGKLYEYSTDSCMDKILDRYERYSYAEKVLISVESETQGNWCHEYRKLKAKVETIQKCQKHLMGEDLETLNLKELQQLEQQLESSLKHIRTRKSQLMLESISELQRKEKSLQEENKVLQKELAEKQKAQRKQVQWGQTQQQTSSSSSCFMIREAAPTTNISIFPVAAGGRLVEGAAAQPQARVGLPPWMLSHLSS

SEQ ID NO: 8 - DNA, 738 bp, zea mays
atggggcgcggtaaggtgcagctgaagcggatcgagaacaagatcaaccgccaggtgaccttctccaagcgccgctcggggctgctcaagaaggcgcacgagatctccgtgctctgcgacgccgaggtcgcgctcatcatcttctccaccaaagggaagctctacgagtattccaccgattcatgtatggacaaaattcttgaccggtacgagcgctactcctatgcagaaaaggttcttatttcagtagaatctgaaactcagggcaattggtgccacgagtatagaaaactaaaggcgaaggtcgagacaatacaaaaatgtcaaaagcacctcatgggagaggatcttgaaacgttgaatctcaaagagcttcagcaactagagcagcagctggagagttcactgaaacatatcagaaccaggaagagccagcttatgctcgagtcaatttcggagctccaacggaaggagaagtcgctgcaggaggagaacaaggttctgcagaaggagctcgcggagaagcagaaagcccagcggaagcaagtgcaatggggccaaacccaacagcagaccagttcgtcttcctcgtgcttcatgataagggaagctgccccaacaacaaatatcagcatttttcctgtggcagcaggcgggaggttggtggaaggtgcagcagcgcagccacaggctcgcgttggactaccaccatggatgcttagccacctgagcagctga

SEQ ID NO: 9 - protein, 202 aa, oryza sativa
MVRGKVQMRRIENPVHRQVTFCKRRGGLLKKARELSVLCDADVGVIIFSSQGKLHELATNGNMHNLVERYQSNVAGGQMEPGALQRQQVAEQGIFLLREEIDLLQRGLRSTYGGGAGEMTLDKLHALEKGLELWIYQIRTTKMQMMQQEIQFLRNKEGILKEANEMLQEKVKEQQKLYMSLLDLHSQQPTQPMTYGNRFFSI

SEQ ID NO: 10 - protein, 253 aa, oryza sativa
MGRGKVQLKRIENKINRQVTFSKRRSGLLKKANEISVLCDAEVALIIFSTKGKLYEYATDSCMDKILERYERYSYAEKVLISAESDTQGNWCHEYRKLKAKVETIQKCQKHLMGEDLESLNLKELQQLEQQLENSLKHIRSRKSQLMLESINELQRKEKSLQEENKVLQKENPCSFLQLVEKQKVQKQQVQWDQTQPQTSSSSSSFMMREALPTTNISNYPAAAGERIEDVAAGQPQHVRIGLPPWMLSHING

SEQ ID NO: 11 - protein, 265 aa, hordeum vulgare
MGRGPVQLRRIENKINRQVTFSKRRSGLLKKAHEISVLCDAEVALIVFSTKGKLYEYSSQDSSMDVILERYQRYSFEERAVLDPSTGDQANWGDEYGSLKIKLDALQKSQRQLLGEQLDPLTTKELQQLEQQLDSSLKHIRSRKNQLLFESISELQKKEKSLKDQNGVLQKHLVETEKEKNNVLSNIHHREQLNEATNIHHQEQLSGATTSSPSPTPPTAQDSMAPPNIGPYQSRGGGDPEPQPSPAQANNSNLPPWMLRTIGNR

SEQ ID NO: 12 - protein, 246 aa, hordeum vulgare
MGRGRVELKRIENKINRQVTFAKRRNGLLKKAYELSVLCDAEVALIVFSNRGKLYEFCSTQSMTKTLDKYQKCSYAGPETTVQNRENEQLKNSRNEYLKLKTRVDNLQRTQRNLLGEDLDSLGIKELESLEKQLDSSLKHIRTTRTQHMVDQLTELQRREQMFSEANKCLRIKLEESNQVHGQQLWEHNNNVLSYERQPEVQPQMHGGNGFFHPLDAAGEPTLHIGYPPESLNSSCMTTFMPPWLP

SEQ ID NO: 13 - protein, 244 aa, hordeum vulgare
MGRGKVQLKRIENKINRQVTFSKRRSGLLKKAHEISVLCDAEVGLIIFSTKGKLYEFSTESCMDKILERYERYSYAEKVLVSSESEIQGNWCHEYRKLKAKVETIQKCQKHLMGEDLESLNLKELQQLEQQLESSLKHIRARKNQLMHESISELQKKERSLQEENKVLQKELVEKQKAQAAQQDQTQPQTSSSSSSFMMRDAPPVADTSNHPAAAGERAEDVAVQPQVPLRTALPLWMVSHING

SEQ ID NO: 14 - protein, 276 aa, hordeum vulgare
MGRGKVQLKRIENKINRQVTFSKRRNGLLKKAHEISVLCDAEVAVIVFSPKGKLYEYATDSSMDKILERYERYSYAEKALISAESESEGNWCHEYRKLKAKIETIQKCHKHLMGEDLDSLNLKELQQLEQQLESSLKHIRSRKSHLMMESISELQKKERSLQEENKALQKELVERQKAASRQQQLQQQQQQQQMQWEHQAQTQTHTHTQNQPQAQTSSSSSSFMMRDQQAHAPQQNICSYPPVTMGGEATAAAAAPEQQAQLRICLPPWMLSHLNA

SEQ ID NO: 15 - protein, 192 aa, hordeum vulgare
QSMPKTLERYQKCSYGGPDTAIQNKENELVQSSRNEYLKLKARVENLQRTQRNLLGEDLGSLGIKDLEQLEKQLDSSLRHIRSTRTQHMLDQLTDLQRKEQMLSEANKCLRRKLEESSQQMQGQMWEQHAANLLGYDHLRQSPHQQQAQHHGGNGFFHPLDPTTEPTLQIGYTQEQINNACVAASFMPTWLP

SEQ ID NO: 16 - DNA, 270 bp, oriza sativa
gtaagcaagagatagggataaggggaagaggaggaagaaggaggaggtgtagggagaaaccggagcaacctcgaagctagtccaaactagtgggaggttgtctttccggcaagccggagcccggagctatcgatcatcaagctttctaccccgaccgacgaggaagaagacgactgatcaattgatcaaaccgatctctccatagctaggtagacaggaggagaggaggaagaagagggggagaggagacttatcttgatcgatggcgcg

SEQ ID NO: 17 - DNA, 371 bp, oriza sativa
tcgacgtaggcatgatggtagcagatcaacagaatgggcattttagtacagtcccactgttagaagagatcactaacccactgactatactgagtggctattctacttgtaggggctcggagatgggctattccttctaacactaataatggcctgggggatacttgtgttcattactagtgtaatatggttaataatgcttgtgttgctgtttgctttgctattctgatgtaccttatttagacaagttcccgcaggaagtgtcttttagtattgtatttgtcttgggctgtggtgctttgtttttccctaaagaactcttgaggagctctgttgttgaaccatttcaagtaattgagactattgtttcc

(19) United States
(12) Patent Application Publication
(10) Pub. No.: US 2014/0130202 A1
(43) Pub. Date: May 8, 2014

(54) STRESS-RESISTANT PLANTS AND THEIR PRODUCTION

(71) Applicants: Pascal Gantet, Jacou (FR); Emmanuel Guiderdoni, Aniane (FR); Ngangiang Khong, Montpellier (FR); Jean-Benoit Morel, Montpellier (FR)

(72) Inventors: Pascal Gantet, Jacou (FR); Emmanuel Guiderdoni, Aniane (FR); Ngangiang Khong, Montpellier (FR); Jean-Benoit Morel, Montpellier (FR)

(21) Appl. No.: 13/882,734

(22) PCT Filed: Nov. 3, 2011
(86) PCT No.: PCT/EP2011/069367

§ 371 Date: Oct. 15, 2013

Related U.S. Application Data
(60) Provisional application No. 61/410,074, filed on Nov. 4, 2010.

(51) Int. Cl. C12N 15/82; C12N 15/113

(52) U.S. Cl. CPC C12N 15/8271; C12N 15/113; C12N 15/8273; C12N 15/8282; C12N 15/8281
USPC 800/276; 800/298; 800/320; 800/320.1; 800/320.3; 800/320.2; 800/301; 800/278; 800/286; 536/24.5

(57) ABSTRACT

The present invention relates to plant genes involved in negative regulation of resistance to biotic and/or abiotic stress and uses thereof. More particularly, the present invention relates to plants comprising an inactivated MADS-box gene function, and having increased resistance to biotic and/or abiotic stress. The invention also relates to methods for producing modified plants having increased resistance to fungal, bacterial pathogens and/or to drought stress. In particular, the invention relates to methods for producing plants with inactivated MAD26 gene, or an ortholog thereof, and exhibiting resistance to biotic and/or abiotic stress.

### FIELD OF THE INVENTION

The present invention relates to plant genes involved in negative regulation of resistance to biotic and/or abiotic stress and uses thereof. More particularly, the present invention relates to plants comprising an inactivated MADS-box gene function, and having increased resistance to biotic and/or abiotic stress. The invention also relates to methods for producing modified plants having increased resistance to fungal, bacterial pathogens and/or to drought stress. In particular, the invention relates to methods for producing plants with inactivated MAD26 gene, or an ortholog thereof, and exhibiting resistance to biotic and/or abiotic stress.

### BACKGROUND OF THE INVENTION

Crop plants are continuously confronted with diverse pathogens. In particular, infection of crop plants with bacteria and fungi can have a devastating impact on agriculture due to loss of yield and contamination of plants with toxins. Other factors that cause drastic yield reduction in most crops are abiotic stress factors such as drought, salinity, heavy metals and temperature.

According to FAO estimates, diseases, insects and weeds cause as much as 25% yield losses annually in cereal crops (Khush, 2005). For example, in China alone, it is estimated that 1 million hectares are lost annually because of blast disease (Khush and Jena 2009). Between 1987 and 1996, fungicides represented, for example, up to 20 and 30% of the culture costs in China ($46 Million) and Japan ($461 Million) respectively.

To meet the increasing demand on the world food supply, it will be necessary to produce up to 40% more rice by 2030 (Khush 2005). This will have to be on a reduced sowing area due to urbanization and increasing environmental pollution. For example, the sowing area in China decreased by 8 million hectares between 1996 and 2007. Improvement of yield per plant is not the only way to achieve this goal; reduction of losses by biotic and abiotic stress is also a solution.

One of the most devastating fungal diseases is a blast disease, which is caused by the ascomycete Magnaporthe oryzae, also known as rice blast fungus. Members of the M. grisea/M. oryzae complex (containing at least two biological species: M. grisea and M. oryzae) are extremely effective plant pathogens as they can reproduce both sexually and asexually to produce specialized infectious structures known as appressoria that infect aerial tissues and hyphae that can infect root tissues. Magnaporthe fungi can also infect a number of other agriculturally important cereals including wheat, rye, barley, and pearl millet causing diseases called blast disease or blight disease. Other plant fungal pathogens of economic importance include species fungal pathogens are selected from Puccinia, Aspergillus, Ustilago, Septoria, Erisyphe, Rhizoctonia and Fusarium species. Fusarium contamination in cereals (e.g., barley or wheat) can result in head blight disease. For example, the total losses in the US of barley and wheat crops between 1991 and 1996 have been estimated at $3 billion (Brewing Microbiology, 3rd edition. Priest and Campbell, ISBN 0-306-47288-0).

Other devastating for agriculture plant pathogens are bacterial pathogens from Xanthomonas, Ralstonia, Erwinia, Pectobacterium, Pantoea, Agrobacterium, Pseudomonas, Burkholderia, Acidovorax, Clavibacter, Streptomyces, Xylella, Spiroplasma and Phytoplasma species. Plant pathogenic bacteria cause many different kinds of symptoms that include galls and overgrowths, wilts, leaf spots, specks and blights, soft rots, as well as scabs and cankers. Some plant pathogenic bacteria produce toxins or inject special proteins that lead to host cell death or produce enzymes that break down key structural components of plant cells. An example is the production of enzymes by soft-rotting bacteria that degrade the pectin layer that holds plant cells together. Still others, such as Ralstonia spp., colonize the water-conducting xylem vessels causing the plants to wilt and die. Agrobacterium species even have the ability to genetically modify or transform their hosts and bring about the formation of cancer-like overgrowths called crown gall. Bacterial diseases in plants are difficult to control. Emphasis is on preventing the spread of the bacteria rather than on curing the plant.

Cultural practices can either eliminate or reduce sources of bacterial contamination, such as crop rotation to reduce over-wintering. However, the most important control procedure is ensured by genetic host resistance providing resistant varieties, cultivars, or hybrids.

Pathogen infection of crop plants can have a devastating impact on agriculture due to loss of yield and contamination of plants with toxins. Currently, outbreaks of blast disease are controlled by applying expensive and toxic fungicidal chemical treatments using for example probenazole, tricyclazole, pyroquilon and phthalide, or by burning infected crops. These methods are only partially successful since the plant pathogens are able to develop resistance to chemical treatments.

To reduce the amount of pesticides used, plant breeders and geneticists have been trying to identify disease resistance loci and exploit the plant's natural defense mechanism against pathogen attack. Plants can recognize certain pathogens and activate defense in the form of the resistance response that may result in limitation or stopping of pathogen growth. Many resistance (R) genes, which confer resistance to various plant species against a wide range of pathogens, have been identified. However, most of these R genes are usually not durable since pathogens can easily breakdown this type of resistance.

Consequently, there exists a high demand for novel efficient methods for controlling plant diseases, as well as for producing plants of interest with increased resistance to biotic and abiotic stress.

### SUMMARY OF THE INVENTION

The present invention provides novel and efficient methods for producing plants resistant to biotic and abiotic stress. Surprisingly, the inventors have discovered that mutant plants with a defective MADS-box gene are resistant to plant diseases. In particular, the inventors have demonstrated that MAD26 gene is a negative regulator of biotic stress response, and that plants with a defective MAD26 gene are resistant to fungal and bacterial pathogens while plants over-expressing the MAD26 gene are more susceptible to plant diseases. Moreover, the inventors have shown that inhibiting MAD26 gene expression increases plant resistance to drought stress. To our knowledge, this is the first example of regulation of biotic and abiotic resistance in plants by a transcription factor of the MADS-box family. In addition, the inventors have identified orthologs of MAD26 in various plants, as well as other members of the MADS-box gene family, thus extending the application of the invention to different cultures and modifications.

An object of this invention therefore relates to plants comprising a defective MADS-box transcription factor function. As will be discussed, said plants exhibit an increased or improved resistance to biotic and/or abiotic stress. Preferably, said plants are monocots. More preferably, said plants are cereals selected from the Poaceae family (e.g., rice, wheat, barley, oat, rye, sorghum or maize).

The invention more particularly relates to plants having a defective MADS-box protein and exhibiting an increased resistance to biotic and/or abiotic stress.

Another particular object of this invention relates to plants comprising a defective MADS-box gene and exhibiting an increased resistance to biotic and/or abiotic stress.

A further object of this invention relates to seeds of plants of the invention, or to plants, or descendents of plants grown or otherwise derived from said seeds.

A further object of the invention relates to a method for producing plants having increased resistance to biotic and/or abiotic stress, wherein the method comprises the following steps:
(a) inactivation of a MADS-box gene or protein, preferably a MAD26 gene or protein, or an ortholog thereof, in a plant cell;
(b) optionally, selection of plant cells of step (a) with inactivated MADS-box gene or protein;
(c) regeneration of plants from cells of step (a) or (b); and
(d) optionally, selection of a plant of (c) with increased resistance to and biotic and/or abiotic stress, said plant having a defective MADS-box gene or protein, preferably a defective MAD26 gene or protein, or an ortholog thereof.

As will be further disclosed in the present application, the MADS-box transcription factor function may be rendered defective by various techniques such as, for example, by inactivation of the gene (or RNA), inactivation of the protein, or inactivation of the transcription or translation thereof. Inactivation may be accomplished by, e.g., deletion, insertion and/or substitution of one or more nucleotides, site-specific mutagenesis, ethyl methanesulfonate (EMS) mutagenesis, targeting induced local lesions in genomes (TILLING), knock-out techniques, or gene silencing using, e.g., RNA interference, ribozymes, antisense, aptamers, and the like. The MADS-box function may also be rendered defective by altering the activity of the MADS-box protein, either by altering the structure of the protein, or by expressing in the cell a ligand of the protein, or an inhibitor thereof, for instance.

The invention also relates to a method for conferring or increasing resistance to biotic and/or abiotic stress to a plant, comprising a step of inhibiting, permanently or transiently, a MADS-box function in said plant, e.g., by inhibiting the expression of the MADS-box gene(s) in said plant.

Another object of this invention relates to an inhibitory nucleic acid, such as an RNAi, an antisense nucleic acid, or a ribozyme, that inhibits the expression (e.g., transcription or translation) of a MADS-box gene.

Another object of the invention relates to the use of such nucleic acid for increasing resistance of plants or plant cells to biotic and/or abiotic stress.

A further object of the invention relates to plants transformed with a vector comprising a nucleic acid sequence expressing an inhibitory nucleic acid, such as an RNAi, an antisense, or a ribozyme molecule that inhibits the expression of a MADS-box gene.

The invention is applicable to produce cereals having increased resistance to biotic and/or abiotic stress, and is particularly suited to produce resistant wheat, rice, barley, oat, rye, sorghum or maize.

### BRIEF DESCRIPTION OF THE DRAWINGS

FIG. 1: Constitutive expression of the OsMAD26 gene. QPCR analysis of the expression profile of OsMAD26. A: OsMAD26 expression in different organs from plantlet cultivated in standard condition (MS/2). L: leaf, S: stem, CR: crown root, SR−A: seminal root without apex, SR+A: seminal root apex. B-C, expression patterns of OsMAD26 in shoot (B) and in root (C) of 7 days old rice seedlings cultivated in standard condition (C), with 150 mM NaCl (SS), 100 mM manitol (OS). Values represent the mean obtained from two independent biological repetitions, bars are standard error. *: significant difference with p=0.05.

FIG. 2: Expression vector pANDA used for cloning OsMAD26 cDNA. The pANDA vector allows the expression under the control of the constitutive promoter of ubiquitin gene from maize of the cloned gene sequence tag (GST) in sense and antisense orientation separated by a GUS spacing sequence. The insertion of the GSTs was checked by sequencing. The obtained plasmids were named pANDA-GST1 and pANDA-GST2 (respectively for GST1 and GST2), and were transferred in an A. tumefaciens strain EHA105 for plant transformation.

FIG. 3: Amplification of GST1 and GST2 sequence tags specific of MAD26-cDNA (from root of Oryza sativa) and MAD26-RNAi prediction. A PCR amplification was performed with a couple of specific primers designed in the 5′ and 3′ UTR of OsMADS 26 (PC8 Forward: 5′-aagcaagagatagggataag-3′, PC8 Reverse: 5′-attacttgaaatggttcaac-3′). The amplified cDNA were cloned using the pGEM-T easy cloning kit of Promega. Obtained plasmid was named pGEMT-PC8. From this plasmid further PCR reactions were done using specific primers possessing the recombination sequence for BP recombinase of the gateway cloning technology of Invitrogen in their 5′ end to amplify the OsMAD26 cDNA (PC8 BP forward: 5′-ggggacaagtttgtacaaaaaagcaggctgaagaggaggaagaaggagg-3′ and PC8 BP Reverse: 5′-ggggaccactttgtacaagaaagctgggtgctcctcaagagttctttag-3′), a 215 bp fragment located in the 5′ UTR of OsMAD26, named GST1 (PC8 BP forward and GST1 reverse: 5′-ggggaccactttgtacaagaaagctgggtccctcttcttcctcctctcc-3′) and a 321 bp fragment comprising the end of the last exon and the major part of the 3′ UTR region of OsMAD26, named GST2 (GST2 forward: 5′-ggggacaagtttgtacaaaaaagcaggctcatgatggtagcagatcaac-3′ and PC8 BP reverse).

FIG. 4: MAD26 gene expression pattern in transgenic and RNA-interfered plants using quantitative QPCR analysis. A: OsMAD26 expression levels in overexpressing (dark bars) and correspondent control (white bars) plants cultivated in greenhouse. B: OsMAD26 expression levels in RNA interfered (grey bars) and correspondent control (white bars) plants cultivated in greenhouse. C: OsMAD26 expression levels in RNA interfered (grey bars) and correspondent control (white bars) 7-d-old seedlings cultivated on MS/2 medium added with 125 mM of manitol. Values represent the mean obtained from two independent biological repetitions, bars are standard error.

FIG. 5: MAD26 RNA-interfered plants are more resistant to fungal infection while plants overexpressing the MAD26 gene are less resistant to fungal infection. Resistance of OsMAD26 transgenic lines against Magnaporthe oryzae (M. oryzae). Nine independent rice lines overexpressing (PCA, PCB) (black bars) or interfered (PD1, PD2) (grey bars) OsMAD26 and corresponding control lines transformed with empty vectors (PCO, PDO) and wild-type plants (WT) (white bars) were assayed. A: Symptom severity in leaves of transgenic and control plants inoculated with the GY11 strain of M. oryzae. Photographs were taken at 3 days post inoculation. Maratelli, highly susceptible control. B: Percentage of susceptible versus total lesions observed in M. oryzae-infected leaves at 3 days after inoculation. Values represent the mean obtained from ten inoculated plants for each line, bars are corresponding standard error. Results shown are representative of the data obtained for three independent experiments. *: significant difference with p<0.05; **: significant difference with p<0.01; ***: significant difference with p<0.001.

FIG. 6: MAD26 RNA-interfered plants are more resistant to bacterial infection while plants overexpressing the MAD26 gene are less resistant to bacterial infection. Resistance of OsMAD26 transgenic lines against Xanthomonas oryzae pv. Oryzae (Xoo). Nine independent rice lines overexpressing (PCA, PCB) (black bars) or interfered (PD1, PD2) (grey bars) OsMAD26 and corresponding control lines transformed with empty vectors (PCO, PDO) and wild-type plants (WT) (white bars) were assayed. A: Symptom severity in leaves of transgenic and control plants inoculated with the PDX99 strain of Xoo. Photographs were taken at 14 days post inoculation. B: Length of lesion produced in Xoo-infected leaves at 14 dpi. Values represent the mean obtained from ten inoculated plants for each line, bars are corresponding standard error. Results shown are representative of the data obtained for two independent experiments. *: significant difference with p<0.05; **: significant difference with p<0.01.

FIG. 7: MAD26 induction under osmotic stress. OsMAD26 gene is induced under osmotic stress.

FIG. 8: MAD26 gene expression pattern in transgenic plants. A: OsMAD26 gene is silenced in RNAi-interfered plants (lines 2PD1-A, 2PD1-B, 2PD2-A, 2PD2-B). B: Under osmotic stress, MAD26 gene is still silenced.

FIG. 9: MAD26 RNA-interfered plants are more resistant to drought stress and plants overexpressing the MAD26 gene are less resistant to drought stress. Leaf relative water content kinetics of OsMAD26 transgenic plants during drought stress. Drought stress was applied on twenty days old plants growing in greenhouse in soil pots, by watering stopping. The values represent the mean obtained from five plants by line, bars are standard error. 4PC1, 4PC2: OsMAD26 overexpressing plants, 4PD1A, 4PD2A: OsMAD26 interfered plants, 4PCO, 4PDO: plants transformed with empty vectors, 4WT: untransformed plants.

FIG. 10: MAD26-RNAi silenced plants are more resistant to drought stress. At the 6^{th} leaf stage, plants were not watered any more, and were kept under drought stress conditions during 21 days.

### DETAILED DESCRIPTION OF THE INVENTION

The MADS-box family of genes code for transcription factors which have a highly conserved sequence motif called MADS-box. These MADS box transcription factors have been described to control diverse developmental processes in flowering plants, ranging from root to flower and fruit development (Rounsley et al., 1995). The N-terminal part of the encoded factor seems to be the major determinant of DNA-binding specificity and the C-terminal part seems to be necessary for dimerisation.

There are several reported members of the MADS-box family of genes, including MAD26, MAD33 and MAD14.

MAD26 gene, the rice ortholog of AGL12 in Arabidopsis thaliana, was recently proposed to be involved in senescence or maturation processes since MAD26 transcript level was increased in an age-dependent manner in leaves and roots (Lee et al., 2008). However MAD26 knock-out rice plants, which were tested under various stress conditions (such as drought, high salt, and stress mediators), showed no difference in comparison with wild-type plants.

Surprisingly, the inventors have now shown that plants with inactivated MAD26 gene are more resistant to abiotic stress such as drought stress. Moreover, the inventors have also discovered that MAD26 is a negative regulator of plant resistance to pathogens, i.e., its inhibition increases resistance. This is the first example of regulation of resistance in plants by a transcription factor of the MADS-box family. MADS-box genes thus represent novel and highly valuable targets for producing plants of interest with increased resistance to pathogens.

The present invention thus relates to methods for increasing pathogen resistance in plants based on a regulation of MADS-box gene function, in particular of MAD26 gene function.

The invention also relates to plants or plant cells having an inactivated MADS-box gene function, preferably MAD26 gene function, or an ortholog thereof.

The invention also relates to constructs (e.g., nucleic acids, vectors, cells, etc) suitable for production of such plants and cells, as well as to methods for producing plant resistant regulators.

The present disclosure will be best understood by reference to the following definitions:

### DEFINITIONS

As used therein, the term “MADS-box protein” designates proteins containing a MADS-box amino acid sequence and which have a transcription factor activity. Typical MADS-box proteins bind to a DNA consensus sequence CC(A/T)₄NNGG (wherein N represents any nucleotide base), or an homogous sequence thereof. Preferred MADS-box proteins comprise the following amino acid sequence IXXXXXXXXTXXKRXXGXXKKXXEXXXL (wherein X represents any amino acid). Specific examples of a MADS-box protein include, without limitation, MAD26, MAD33 or MAD14 proteins. MADS-box have been isolated or identified in various plant species. Specific examples of MADS-box proteins include Oryza sativa MADS-box proteins comprising a sequence selected from SEQ ID NOs: 2, 9, or 10, Triticum aestivum MADS-box protein comprising a sequence of SEQ ID NO: 3, and Hordeum vulgare MADS-box proteins comprising a sequence selected from SEQ ID NOs: 11, 12, 13, 14 or 15. The term MADS-box proteins also encompass any variant (e.g., polymorphism) of a sequence as disclosed above, as well as orthologs of such sequences in distinct plant species.

Within the context of the present invention, the term “MADS-box gene” designates any nucleic acid that codes for a MADS-box protein as defined above. The term “MADS-box gene” includes MADS-box DNA (e.g., genomic DNA) and MADS-box RNA (e.g., mRNA). Examples of MADS-box genes include a MAD26, MAD33 or MAD14 DNA or RNA of Oryza sativa, Triticum aestivum, Hordeum vulgare, Zea mays, Sorghum bicolor, Arabidopsis thaliana. Specific example of a MADS-box gene comprises the nucleic acid sequence of SEQ ID NOs: 1, 4, 6 or 8.

In the most preferred embodiment, a MADS-box gene is a MAD26 gene, a MAD33 gene, a MAD14 gene, or orthologs thereof. Within the context of the present invention, the term “ortholog” designates a related gene or protein from a distinct species, having a level of sequence identity to a reference MADS-box gene above 50% and a MADS-box gene like activity. An ortholog of a reference MADS-box gene is most preferably a gene or protein from a distinct species having a common ancestor with said reference MADS-box gene, acting as a negative regulator of plant resistance to biotic and/or abiotic stress, and having a degree of sequence identity with said reference MADS-box gene superior to 50%. Preferred orthologs of a reference MADS-box gene have least 60%, preferably at least 70%, most preferably at least 70, 80, 90, 95% or more sequence identity to said reference sequence, e.g., to the sequence shown in SEQ ID NO: 1 (Oryza sativa). MADS-box gene orthologs can be identified using such tools as “best blast hit” searches or “best blast mutual hit” (BBMH). MAD26 orthologs have been identified by the inventors in various plants, including wheat, barley, sorghum or maize (see Table 2 and sequence listing). Specific examples of such orthologs include the nucleic acid sequence of SEQ ID NO: 4, 6 or 8, and the amino acid sequence of SEQ ID NO: 3, 5 or 7.

Further examples of MADS-box genes or proteins are listed below:

Rice (Oryza sativa)

### GenBank:

Os12g10520.1
Os12g10520.2
Os03g54160.1
Os03g54160.2
Os07g41370.1
Os07g01820.3
Os07g01820.2
Os06g06750.1
Os07g01820.4
Os01g66290.2
Os01g66290.1
Os03g11614.1
Os03g03100.1
Os02g45770.1
Os01g52680.1
Wheat (Triticum aestivum)

### GenBank:

### CAM59056

### AM502878.1

### DQ512350.1

### AM502870.1

### DQ534490.1

### DQ512331.1

### AM502886.1

### AM502877.1

### DQ512370.1

### DQ512334.1

### AM502867.1

### AB295661.1

### AB295660.1

### AB295659.1

### DQ512345.1

### AM502903.1

### DQ534492.1

### DQ512347.1

### AM502868.1

### DQ512351.1

### AB295664.1

### DQ512356.1

### DQ512348.1

### AM502901.1

### AM502900.1

Maize (Zea mays)

### GenBank:

### ACG41656.1

### ACR35354.1

### NP_{—}001148873.1

Sorghum (Sorghum bicolor)

### GenBank:

### XP_{—}002443744.1

Within the context of the present invention, the term “biotic stress” designates a stress that occurs as a result of damage done to plants by living organism, e.g. plant pathogens. The term “pathogens” designates all pathogens of plants in general such as bacteria, viruses, fungi, parasites or insects. More preferably the pathogens are fungal and/or bacterial pathogens. In a particular embodiment, fungal pathogens are cereal fungal pathogens. Examples of such pathogens include, without limitation, Magnaporthe, Puccinia, Aspergillus, Ustilago, Septoria, Erisyphe, Rhizoctonia and Fusarium species. In the most preferred embodiment, the fungal pathogen is Magnaporthe oryzae.

In another particular embodiment, bacterial pathogens are cereal bacterial pathogens. Examples of such pathogens include, without limitation, Xanthomonas, Ralstonia, Erwinia, Pectobacterium, Pantoea, Agrobacterium, Pseudomonas, Burkholderia, Acidovorax, Clavibacter, Streptomyces, Xylella, Spiroplasma and Phytoplasma species. In the most preferred embodiment, the bacterial pathogen is Xanthomonas oryzae.

Within the context of the present invention, the term “abiotic stress” designates a stress that occurs as a result of damage done to plants by non-living environmental factors such as drought, extreme cold or heat, high winds, salinity, heavy metals.

The invention is particularly suited to create cereals resistant to Magnaporthe and/or Xanthomonas and/or resistant to drought stress. Preferably, the cereal is selected from rice, wheat, barley, oat, rye, sorghum or maize. In the most preferred embodiment the resistant cereal is rice, for example Oryza sativa indica, Oryza sativa japonica.

Different embodiments of the present invention will now be further described in more details. Each embodiment so defined may be combined with any other embodiment or embodiments unless otherwise indicated. In particular, any feature indicated as being preferred or advantageous may be combined with any other feature or features indicated as being preferred or advantageous.

### MADS-Box Function-Defective Plants

As previously described, the present invention is based on the finding that MAD26 gene is a negative regulator of plant resistance to biotic and/or abiotic stress. The inventors have demonstrated that the inactivation of MAD26 gene increases plant resistance to fungal pathogens, bacterial pathogens and to drought stress.

The present invention thus relates to methods for increasing pathogen resistance and abiotic stress resistance in plants, based on a regulation of MADS-box transcription factor pathways.

The invention also relates to plants or plant cells having a defective MADS-box function.

The invention also relates to constructs (e.g., nucleic acids, vectors, cells, etc) suitable for production of such plants and cells, as well as to methods for producing plant resistant regulators.

According to a first embodiment, the invention relates to a plant or a plant cell comprising a defective MADS-box function. The term “MADS-box function” indicates any activity mediated by a MADS-box protein in a plant cell. The MADS-box function may be effected by the MADS-box gene expression or the MADS-box protein activity.

Within the context of this invention, the terms “defective”, “inactivated” or “inactivation”, in relation to MADS-box function, indicate a reduction in the level of active MADS-box protein in the cell or plant. Such a reduction is typically of about 20%, more preferably 30%, as compared to a wild-type plant. Reduction may be more substantial (e.g., above 50%, 60%, 70%, 80% or more), or complete (i.e., knock-out plants).

Inactivation of MADS-box function may be carried out by techniques known per se in the art such as, without limitation, by genetic means, enzymatic techniques, chemical methods, or combinations thereof. Inactivation may be conducted at the level of DNA, mRNA or protein, and inhibit the expression of the MADS-box gene (e.g., transcription or translation) or the activity of MADS-box protein.

Preferred inactivation methods affect expression and lead to the absence of production of a functional MADS-box protein in the cells. It should be noted that the inhibition of MADS-box function may be transient or permanent.

In a first embodiment, defective MADS-box gene is obtained by deletion, mutation, insertion and/or substitution of one or more nucleotides in one or more MADS-box gene(s). This may be performed by techniques known per se in the art, such as e.g., site-specific mutagenesis, ethyl methanesulfonate (EMS) mutagenesis, targeting induced local lesions in genomes (TILLING), homologous recombination, conjugation, etc.

The TILLING approach according to the invention aims to identify SNPs (single nucleotide polymorphisms) and/or insertions and/or deletions in a MADS-box gene from a mutagenized population. It can provide an allelic series of silent, missense, nonsense, and splice site mutations to examine the effect of various mutations in a gene.

Another particular approach is gene inactivation by insertion of a foreign sequence, e.g., through transposon mutagenesis using mobile genetic elements called transposons, which may be of natural or artificial origin.

According to another preferred embodiment, the defective MADS-box function is obtained by knock-out techniques.

In the most preferred embodiment, the defective MADS-box function is obtained by gene silencing using RNA interference, ribozyme or antisense technologies. Within the context of the present invention, the term “RNA interference” or “RNAi” designates any RNAi molecule (e.g. single-stranded RNA or double-stranded RNA) that can block the expression of MADS-box genes and/or facilitate mRNA degradation by hydridizing with the sequences of MADS-box mRNA.

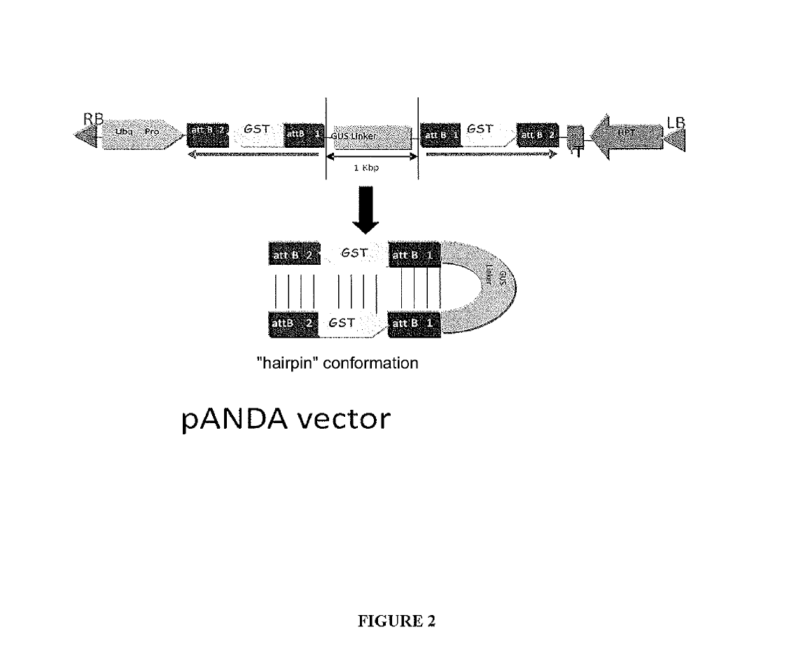

In a particular embodiment, an inhibitory nucleic acid molecule which is used for gene silencing comprises a sequence that is complementary to a sequence common to several MADS-box genes or RNAs. Such a sequence may, in particular, encode the MAD-box motif. In a preferred embodiment, such an inhibitory nucleic acid molecule comprises a sequence that is complementary to a sequence present in a MAD26 gene and that inhibits the expression of a MAD26 gene. In a particular embodiment, such an RNAi molecule comprises a sequence that is complementary to a sequence of the MAD26 gene comprising the GST1 or GST2 sequence. In a preferred embodiment, such an RNAi molecule comprises a sequence producing a hairpin structure RNAi-GST1 or RNAi-GST2 (FIG. 2; SEQ ID NO: 16 and 17). In another particular embodiment, such an inhibitory nucleic acid molecule comprises a sequence that is complementary to a sequence present in a MAD33 or MAD 14 gene and that inhibits the expression of said MAD33 or MAD14 gene.

As illustrated in the examples, MAD26 interfered plants are still viable, show no aberrant developmental phenotype, and exhibit increased resistance to plant pathogens and to drought stress.

MADS-box protein synthesis in a plant may also be reduced by mutating or silencing genes involved in the MADS-box protein biosynthesis pathway. Alternatively, MADS-box protein synthesis and/or activity may also be manipulated by (over)expressing negative regulators of MADS-box transcription factors. In another embodiment, a mutant allele of a gene involved in MADS-box protein synthesis may be (over)expressed in a plant.

MADS-box function inactivation may also be performed transiently, e.g., by applying (e.g., spraying) an exogenous agent to the plant, for example molecules that inhibit MADS-box protein activity.

Preferred inactivation is a permanent inactivation produced by destruction of one or more MADS-box genes, e.g., by deletion or by insertion of a foreign sequence of a fragment (e.g., at least 50 consecutive bp) of the gene sequence.

In a specific embodiment, more than one defective MADS-box gene(s) are obtained by knock-out techniques.

In another embodiment, defective MADS-box function is obtained at the level of the MADS-box protein. For example, the MADS-box protein may be inactivated by exposing the plant to, or by expressing in the plant cells e.g., regulatory elements interacting with MADS-box proteins or specific antibodies.

Thus, the MADS-box function in plant resistance may be controlled at the level of MADS-box gene, MADS-box mRNA or MADS-box protein.

In a variant, the invention relates to a plant with increased resistance to biotic and/or abiotic stress, wherein said plant comprises an inactivated MAD26, MAD33, or MAD14 gene, or an ortholog thereof. In another preferred embodiment, several MADS-box genes present in the plant are defective.

In another variant, the invention relates to a plant with increased resistance to biotic and/or abiotic stress, wherein said plant comprises at least one inactivated MAD-box protein, e.g. MAD26, MAD33 or MAD14 protein.

In another variant, the invention relates to a plant with increased resistance to biotic and/or abiotic stress, wherein said increased resistance is due to inactivation of a MAD-box transcription factor mRNA, preferably MAD26, MAD33 or MAD14 mRNA.

In another embodiment, the invention relates to transgenic plants or plant cells which have been engineered to be (more) resistant to biotic and/or abiotic stress by inactivation of MAD-box protein function. In a particular embodiment, the modified plant is a loss-of-function MAD26, MAD33 or MAD14 mutant plant, with increased resistance to biotic and/or abiotic stress.

The invention also relates to seeds of plants of the invention, as well as to plants, or descendents of plants grown or otherwise derived from said seeds, said plants having an increased resistance to pathogens.

The invention also relates to vegetal material of a plant of the invention, such as roots, leaves, flowers, callus, etc.

### Producing of MAD-Box Transcription Factor Defective Resistant Plants

The invention also provides a method for producing plants having increased resistance to biotic and/or abiotic stress, wherein the method comprises the following steps:
(a) inactivation of a MADS-box gene function in a plant cell;
(b) optionally, selection of plant cells of step (a) with inactivated MADS-box gene function;
(c) regeneration of plants from cells of step (a) or (b); and
(d) optionally, selection of a plant of (c) with increased resistance to and biotic and/or abiotic stress, said plant having a defective MADS-box gene function.

As indicated above, inactivation of the MADS-box gene can be done using various techniques. Genetic alteration in the MADS-box gene may also be performed by transformation using the Ti plasmid and Agrobacterium infection method, according to protocols known in the art. In a preferred method, inactivation is caused by RNA interference techniques or knock-out techniques.

According to another preferred embodiment, MADS-box transcription factor defective resistant plants are obtained by transforming plant cells with a recombinant vector expressing an RNAi molecule that silences MADS-box gene(s). Preferably, such a recombinant vector contains a gene sequence tag (GST) specific of nucleic acid sequence encoding a MAD-box transcription factor. In a particular embodiment, such an expression vector contains a sequence tag of SEQ ID NO: 16 (GST1) or a sequence tag of SEQ ID NO: 17 (GST2) which are both specific of MADD26-cDNA sequence. In a preferred embodiment, the recombinant expression vector is pANDA::MAD26, preferably pANDA-GST1 or pANDA-GST2. Typically, the expressed molecule adopts a hairpin conformation and stimulates generation of RNAi against the sequence tag, e.g. GST1 or GST2.

In the most preferred embodiment, resistant plants of the invention comprise a nucleic acid sequence expressing an RNAi molecule that inhibits the expression of a MAD26 gene, and exhibit an increased resistance to biotic and/or abiotic stress. Such a plant can produce RNAi molecules as described above.

The invention also relates to an isolated cDNA comprising a nucleic acid sequence selected from:
(a) a nucleic acid sequence selected from a nucleic acid sequence which encodes a MAD26 transcription factor or an ortholog thereof, or a fragment thereof;
(b) a nucleic acid sequence of SEQ ID NO: 1, 4, 6 or 8, or a fragment thereof;
(c) a nucleic acid sequence which hybridizes to the sequence of (a) or (b) under stringent conditions, and encodes a MAD26 transcription factor or an ortholog thereof; and
(d) a mutant of a nucleic acid sequence of (a), (b) or (c).

Stringent hybridization/washing conditions are well known in the art. For example, nucleic acid hybrids that are stable after washing in 0.1×SSC, 0.1% SDS at 60° C. It is well known in the art that optimal hybridization conditions can be calculated if the sequence of the nucleic acid is known. Typically, hybridization conditions can be determined by the GC content of the nucleic acid subject to hybridization. Typically, hybridization conditions uses 4-6×SSPE (20×SSPE contains Xg NaCl, Xg NaH2PO4 H2O and Xg EDTA dissolved to 1 l and the pH adjusted to 7.4); 5-10×Denhardts solution (50×Denhardts solution contains 5 g Ficoll), 5 g polyvinylpyrrolidone, 5 g bovine serum albumen; X sonicated salmon/herring DNA; 0.1-1.0% s sodium dodecyl sulphate; optionally 40-60% deionised formamide. Hybridization temperature will vary depending on the GC content of the nucleic acid target sequence but will typically be between 42-65° C.

The present invention also relates to a recombinant vector comprising a nucleic acid molecule as described above. Such a recombinant vector may be used for transforming a cell or a plant in order to increase plant resistance to fungal pathogens, or to screen modulators of resistance. Suitable vectors can be constructed, containing appropriate regulatory sequences, including promoter sequences, terminator fragments, polyadenylation sequences, enhancer sequences, marker genes and other sequences as appropriate. Preferably the nucleic acid in the vector is under the control of, and operably linked to an appropriate promoter or other regulatory elements for transcription in a host cell such as a microbial, (e.g. bacterial), or plant cell. The vector may be a bi-functional expression vector which functions in multiple hosts. In a preferred aspect, the promoter is a constitutive or inducible promoter.

### Selecting of Resistant Plants

Selection of plant cells having a defective MADS-box gene can be made by techniques known per se to the skilled person (e.g., PCR, hybridization, use of a selectable marker gene, protein dosing, western blot, etc.).

Plant generation from the modified cells can be obtained using methods known per se to the skilled worker. In particular, it is possible to induce, from callus cultures or other undifferentiated cell biomasses, the formation of shoots and roots. The plantlets thus obtained can be planted out and used for cultivation. Methods for regenerating plants from cells are described, for example, by Fennell et al. (1992) Plant Cell Rep. 11: 567-570; Stoeger et al (1995) Plant Cell Rep. 14: 273-278.

The resulting plants can be bred and hybridized according to techniques known in the art. Preferably, two or more generations should be grown in order to ensure that the genotype or phenotype is stable and hereditary.

Selection of plants having an increased resistance to biotic and/or abiotic stress can be done by applying the pathogen to the plant or exposing a plant to abiotic stress factors, determining resistance and comparing to a wt plant.

Within the context of this invention, the term “increased resistance” to biotic and/or abiotic stress means a resistance superior to that of a control plant such as a wild type plant, to which the method of the invention has not been applied. The “increased resistance” also designates a reduced, weakened or prevented manifestation of the disease symptoms provoked by a pathogen or an abiotic stress factor. The disease symptoms preferably comprise symptoms which directly or indirectly lead to an adverse effect on the quality of the plant, the quantity of the yield, its use for feeding, sowing, growing, harvesting, etc. Such symptoms include for example infection and lesion of a plant or of a part thereof (e.g., different tissues, leaves, flowers, fruits, seeds, roots, shoots), development of pustules and spore beds on the surface of the infected tissue, maceration of the tissue, accumulation of mycotoxins, necroses of the tissue, sporulating lesions of the tissue, colored spots, etc. Preferably, according to the invention, the disease symptoms are reduced by at least 5% or 10% or 15%, more preferably by at least 20% or 30% or 40%, particularly preferably by 50% or 60%, most preferably by 70% or 80% or 90% or more, in comparison with the control plant.

The term “increased resistance” of a plant to biotic and/or abiotic stress also designates a reduced susceptibility of the plant towards infection with plant pathogens and/or towards damage of the plant caused by an abiotic stress factor, or lack of such susceptibility. The inventors have demonstrated, for the first time, a correlation between expression of a MADS-box gene and susceptibility towards infection. As shown in the experimental part, the overexpression of MAD26 gene promotes disease, whereas the MAD26-RNA interference increases resistance. The inventors have therefore proposed that the MADS-box transcription factor signaling increases susceptibility of plants to infection and favors the development of the disease due to biotic and/or abiotic factors.

Preferred plants or cells of the invention are MADS-box RNA interfered plants, preferably MAD26, MAD 33 or MAD 14 RNA interfered plants.

In the most preferred embodiment, the method of the invention is used to produce monocot plants having a defective MAD-box gene, preferably MAD26 gene, with increased resistance to fungal, bacterial pathogens and/or to drought stress. Examples of such plants and their capacity to resist pathogens and drought are disclosed in the experimental section.

Further aspects and advantages of the invention are provided in the following examples, which are given for purposes of illustration and not by way of limitation.

### EXAMPLES

### 1. Materials and Methods—Plant Material and Culture Conditions

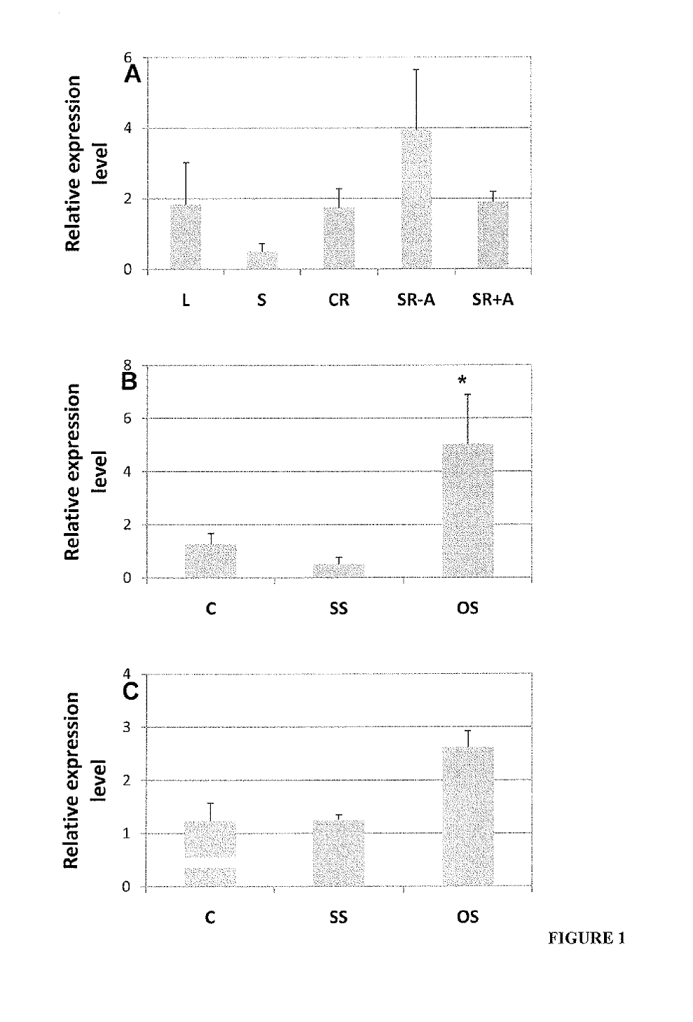

All experiments were done with Oryza sativa japonica, cv ‘Nipponbare. For seedlings obtaining, rice seeds were dehulled and surface disinfected by immersion in 70% ethanol for 1 min, rinsed with sterile distilled water and treated with 3.84% solution of sodium hypochlorite in 30 nm. Finally seeds were rinsed five times with sterile distilled water. Seeds were incubated in sterile distilled water in growth chamber (16 h of light per day, 500 μE m⁻² s⁻¹, 28° C./25° C. day/night) for 2 days. Seeds were transferred in rectangular dishes (245 mm×245 mm, Corning, USA, 7 seeds per dish) containing 250 ml of half Muashige and Skoog (Duchefa) standard medium (MS/2) solidified by 8 g/L of agar type II (Sigma). Theses dishes were transferred and placed vertically in growth chamber. After 7 days of culture, seedlings organs were sampled and used for RT-QPR. Saline and osmotic stresses were applied by adding in the culture medium 150 mM NaCl (Duchefa) or 100 mM manitol (Duchefa), respectively (see FIG. 1). Plants were cultured in soil pots (3 L, Tref, EGO 140 www.Trefgroup.com) in containment greenhouse (16-h-light/8-h-dark cycles, at 28° C. to 30° C.). For plant growth phenotyping, the plants belonging to the different lines were randomly arranged in the greenhouse to avoid position effect on plant growth. Twenty days after germination (DAG), plant height identified from stem base to tip of the top-most leaf on the main tiller and tiller number were measured one time per week until flowering beginning. The flowering beginning was defined as the date when the first spikelet appeared on the plant. The flowering date records the date when spikelets were observed on 50% of the tillers of the plant. After harvesting, the dry weight of the whole plant part, except the root were determined after drying the plants at 70° C. for 96 h. All panicles of each plant were also weighted after dried at 37° C. for 3 days. Then the percentage of seed fertility and the weight of 1000 seeds were measured on the main panicle. This experiment was repeated two times with three plants per line. Statistical analysis of data obtained in these experiments was performed using the ANOVA test with a confidence level of 5%. Specific culture conditions used for pathogen and drought resistance tests are detailed in the corresponding sections.

### 2. Plasmid Construction for Plant Transformation

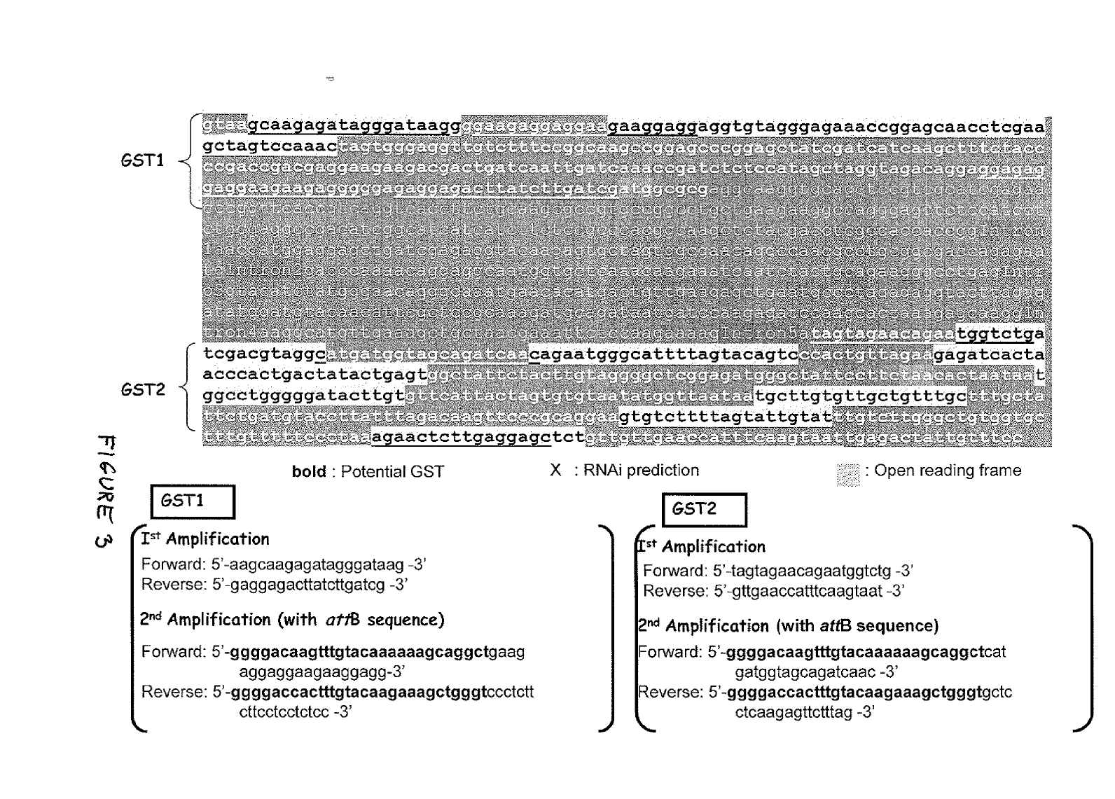

The isolation of OsMAD26 (Os08g02070) cDNA was done by RT-PCR. Total RNA were extracted from 100 mg of 7 day old seedlings grounded in liquid nitrogen using 1 ml of TRIzol (Invitrogen) following the recommendation of the supplier. RNA (20 μg) was incubated with 1 unit of DNase RQ1 (Promega), 1.4 units of RNAsin (Promega) and 20 mM MgCl2 in RNAse-free sterile water, for 30 min at 4° C. RNA (2 μg) was denatured for 5 min at 65° C. and reverse-transcribed with 22.5 μM of oligodT(15) primer (Promega), with 10 u of AMV reverse transcriptase (Promega) for 90 min at 42° C. A PCR amplification was performed with a couple of specific primers designed in the 5′ and 3′ UTR of OsMADS 26 (PC8 Forward: 5′-aagcaagagatagggataag-3′, PC8 Reverse: 5′-attacttgaaatggttcaac-3′). The amplified cDNA were cloned using the pGEM-T easy cloning kit of Promega. Obtained plasmid was named pGEMT-PC8. From this plasmid further PCR reactions were done using specific primers (see FIG. 3) possessing the recombination sequence for BP recombinase of the gateway cloning technology of Invitrogen in their 5′ end to amplify the OsMAD26 cDNA (PC8 BP forward: 5′-ggggacaagtttgtacaaaaaagcaggctgaagaggaggaagaaggagg-3′ and PC8 BP Reverse: 5′-ggggaccactttgtacaagaaagctgggtgctcctcaagagttctttag-3′), a 215 bp fragment located in the 5′ UTR of OsMAD26, named GST1 (PC8 BP forward and GST1 reverse: 5′-ggggaccactttgtacaagaaagctgggtccctcttcttcctcctctcc-3′) and a 321 bp fragment comprising the end of the last exon and the major part of the 3′ UTR region of OsMAD26, named GST2 (GST2 forward: 5′-ggggacaagtttgtacaaaaaagcaggctcatgatggtagcagatcaac-3′ and PC8 BP reverse) (see FIG. 3). PCR cycling conditions were: 94° C. for 4 min (1 cycle) and 94° C. for 1 min, an annealing step at various temperatures depending on the Tm of the primers used (typically Tm −5° C.), for 1.5 min, and 72° C. for 1 min (35 cycles) with a 5 min final extension step at 72° C. PCR was performed in a final volume of 25 μl with 0.25 u of Taq polymerase in MgC12-free buffer (Promega), 2 mM MgCl2, 200 nM each dNTP, appropriate oligonucleotides (1 μM) and cDNA (2 μl) or pGEMT-PC8 plasmid (10 ng).

The BP tailed OsMAD26 cDNA was cloned with the BP recombinase in a PCAMBIA 5300 overexpression modified binary vector named PC5300.OE (see Table 1) where the ccdb gene surrounded by the BP recombination sites were cloned between the constitutive promoter of ubiquitin gene from maize and the terminator of the nopaline syntase gene from A. tumefaciens. After cloning the presence of the OsMAD26 cDNA was verified by sequencing. The plasmid named PC5300.OE-PC8 was transferred into A. tumefaciens strain EHA105. The BP tailed GST1 and GST2 were cloned by BP recombination in the pDON207 entry plasmid (Invitrogen) and transferred with the LR recombinase (Invitrogen) in the binary plasmid pANDA (Miki and Shimamoto, 2004).

The pANDA vector (see FIG. 2) allows the expression under the control of the constitutive promoter of ubiquitin gene from maize of the cloned GST in sense and antisense orientation separated by a GUS spacing sequence. The expressed molecule adopts a hairpin conformation and stimulates the generation of siRNA against the GST sequence. The insertion of the GSTs was checked by sequencing. The obtained plasmids were named pANDA-GST1 and pANDA-GST2, and were transferred in an A. tumefaciens strain EHA105 for plant transformation.

TABLE 1
List of transgenic lines obtained by the method of the invention,
control lines and cloned vectors.

| Lines | Name | Cloned Vector |
|---|---|---|
| Overexpressing (PC) | PC-A | pCAMBIA5300.OE |
| | PC-B | |
| RNAi (GST1) | PD1-A | pANDA |
| | PD1-B | |
| RNAi (GST2) | PD2-A | pANDA |
| | PD2-B | |
| Empty control | PCO | pCAMBIA5300.OE |
| Empty control | PDO | pANDA |
| Wildtype | WT | |

### 3. Plant Transformation and Selection

Transgenic plants were obtained by co-culture of seed embryo-derived callus with Agrobacterium strain EHA105 carrying the adequate binary plasmids following the procedure detailed in Sallaud et al., (2003). Monolocus and homozygotes lines were selected on the basis of the segregation of the antibiotic resistance gene carried by the TDNA. Antibiotic resistance essays were done on 5 days old seedlings incubated in Petri dishes for five days on Watman 3MM paper imbibed with 6 ml of 0.3 mg (5.69·10⁻⁴M) of hygromicin. The presence and the number of the transgenic constructions in plant genome were analyzed by Southern blot. Total genomic DNA was extracted from 200 mg grounded leaf tissue of transgenic (T0 and T1 generation) and control plants using 900 μl of mixed alkyl trimethyl ammonium bromide (MATAB) buffer (100 mM Tris-HCl, pH 8.0, 1.5 M NaCl, 20 mM EDTA, 2% (w/v) MATAB, 1% (w/v) Polyethylen glycol (PEG) 6000, 0.5% (w/v) Na₂SO₂) and incubated at 72° C. for 1 h. The mixture was then cooled to room temperature for 10 nm, and 900 μl of chloroform: isoamyl alcohol (24:1, v/v) was added. After mixing and sedimentation at 6000 g for 10 nm, the aqueous phase was transferred in a new 1.5 ml Eppendorf tube and 20 U of RNase A were added, the mix was incubated at 37° C. for 30 nm. RNAse A was eliminated by a new treatment with 900 μl of Chloroform:isoamyl alcohol (24:1, v/v) and the genomic DNA was finally precipitated after addition of 0.8 volume of isopropanol to the aqueous phase. To evaluate the number of T-DNA insertions in the genome of transgenic plants, 5 μg of genomic DNA were cleaved overnight at 37° C. with 20 units of SacI or Kpn1 (Biolabs) which cut in only one position the TDNA derived from PC5300.OE or pANDA vectors, respectively. DNA fragments were separated by electrophresis in 0.8% agarose gel with TAE buffer (0.04 M Tris-acetate, 0.001 M EDTA). After incubation for 15×mn in 1 L of 0.25NHCL then in 1 L of 0.4N NaOH for 30 nm, DNA was transferred by capillarity in alkaline conditions (0.4N NaOH) onto a Hybond N+ membrane (Amersham Biosciences). The membranes were prehybridized for 4 h at 65° C. in a buffer containing 50 mM Tris-HCl pH 8, 10 mM EDTA pH 8, 5×SSC, 0.2% SDS (w/v) (Eurobio, France), 1×Denhardt's solution (Denhart 50×, Sigma, ref. 2532) and 50 μg of fragmented salmon sperm DNA. Hybridization was performed overnight at 65° C. in a buffer containing 50 mM Tris-HCl pH 8, 10 mM EDTA pH 8, 5×SSC, 0.2% SDS (w/v) (Eurobio, France), 1×Denhardt's solution (Denhart 50×, Sigma, ref. 2532), 40 μg DNA of fragmented salmon sperm DNA and 10% Dextran sulphate (w/v). To check for TDNA copy numbers 80 ng of a 550 bp fragment of the hygromicin resistance gene hph, labelled with [α-³²P] with the random priming kit (Amersham™ UK) was denaturated 10 nm at 95° C. and added to the hybridization mixture. After hybridization, the membranes were washed at 65° C., for 15 nm in 80 ml of buffer 51 containing 2×SSC, 0.5% SDS (Eurobio, France) (v/v), for 30 nm in 50 ml of buffer S2 containing 0.5×SSC and 0.1% SDS (v/v) and finally for 30 nm in 50 ml of buffer S3 containing 0.1×SSC and 0.1% SDS (v/v). The membranes were put in contact with a radiosensible screen (Amersham Bioscience, “Storage Phosphor Screen unmounted 35×43”, ref. 63-0034-80) for 2-3 days. Revelation was performed with a phosphoimageur scanner (Storm 820, Amersham). In order to check for the complete integration of the constructions allowing OsMAD26 constitutive expression or expression of the hairpin molecules designed with specific OsMAD26 GSTs, plant genomic DNA were cleaved with Kpn1 and BamH1 or Sac1 and Kpn1 respectively. Southern blot were done using [α-³²P] labelled specific probes of ORF8 or GST1 or GST2 depending of the construction (see FIG. 3). The expression of OsMAD26 in selected transgenic lines was analyzed by RT-QPCR.

### 4. Real-Time Quantitative Reverse Transcriptase Polymerase Chain Reaction (RT-qPCR) Analysis

Plant material was collected, immediately frozen in liquid nitrogen, and stored at −80° C. Tissues were ground in liquid nitrogen. Total RNA were extracted from 100 mg grounded tissues with 1 ml of TRIzol (Invitrogen) following the recommendation of the supplier. Total RNA were quantified according to their absorbance at 260 nm with a nanoquant Tecan-Spectrophotometer. Five μg of RNA were treated to remove residual genomic for 30 nm at 37° C. DNA with 5 U of DNAse RQ1 (Promega) and 1 μl of RQ1 RNAse-Free DNAse 10× reaction buffer in a final volume of 10 μl. Then, 1 μl of RQ1 DNAse Stop Solution was added to terminate the reaction and the mix was incubated at 65° C. for 10 nm to inactivate the DNAse. The first strand cDNA synthesis was done in 20 μl of final volume using the kit Superscripts III (Invitrogen) following the manufacturer's instructions. The presence of genomic DNA in sample was checked by a PCR reaction using 1 μl of cDNA as template and primers: Act-F (5′-ggatctctcagcaccttccagc-3′), Act-R (5′-cgatatctggagcaaccaaccaca-3′) designed in two exons surrounding an intron of the actin encoding gene (Os01g73310.1). The PCR was done in a thermocycler Techne (TC-512) as follows: 95° C. for 3 min; 30 to 35 cycles of 95° C. for 30 sec, 60° C. for 1 min, and 72° C. for 1 min; with a final extension at 72° C. for 7 min. The PCR was done with 0.5 U of Taq polymerase in a final volume of 50 μl of the corresponding buffer (Biolab) and 2 mM MgCl₂ (Biolab), 0.08 mM of dNTP (Fermentas) and 0.02 μM of each specific primers. Ten microliters from the 50-μL PCR product was separated on a 1% (w/v) agarose gel in 1×TAE buffer and visualized under UV after staining with (6 drops/L) ethidium bromide. For RT-qPCR analysis of gene expression pattern specific forward (F) and reverse (R) primers were designed to amplify a fragment of 200-400 bp in 3′ untranslated zone (3′-UTR) of each studied gene using the Vector NTI (version 10.1) software with default parameters. The RT-qPCR was performed with LighCycler 480 system (Roche) using the SYBR green master mix (Roche) containing optimized buffer, dNTP and Taq DNA polymerase, and manufactured as described in the user manual. The reaction was carried out in 96-well optical reaction plates (Roche). The reaction mix contained 7.5 μl SYBR Green QPCR Master Mix (Roche), 250 nm of each primer (F and R), and 30 of 10 fold diluted cDNA template. All reactions were heated to 95° C. for 5 min, followed by 45 cycles of 95° C. for 10 s and 60° C. for 30 s. Melt curve analysis and gel electrophoresis of the PCR products were used to confirm the absence of non-specific amplification products. Transcripts from an EP gene (Expressed Protein, Os06g11070.1) were also detected and used as an endogenous control to normalize expression of the other genes. EP was chosen as the housekeeping gene because its expression appeared to be the most stable in different tissues and physiological conditions (Canada et al, 2007). Relative expression level were calculated by subtracting the Cₜ (threshold cycle) values for EP from those of the target gene (to give ΔC_{T}), then ΔΔCₜ and calculating 2^{-ΔΔct} (Giulietti et al. 2001). Reactions were performed in triplicate to provide technical replicates and all experiments were replicated at least once with similar results.

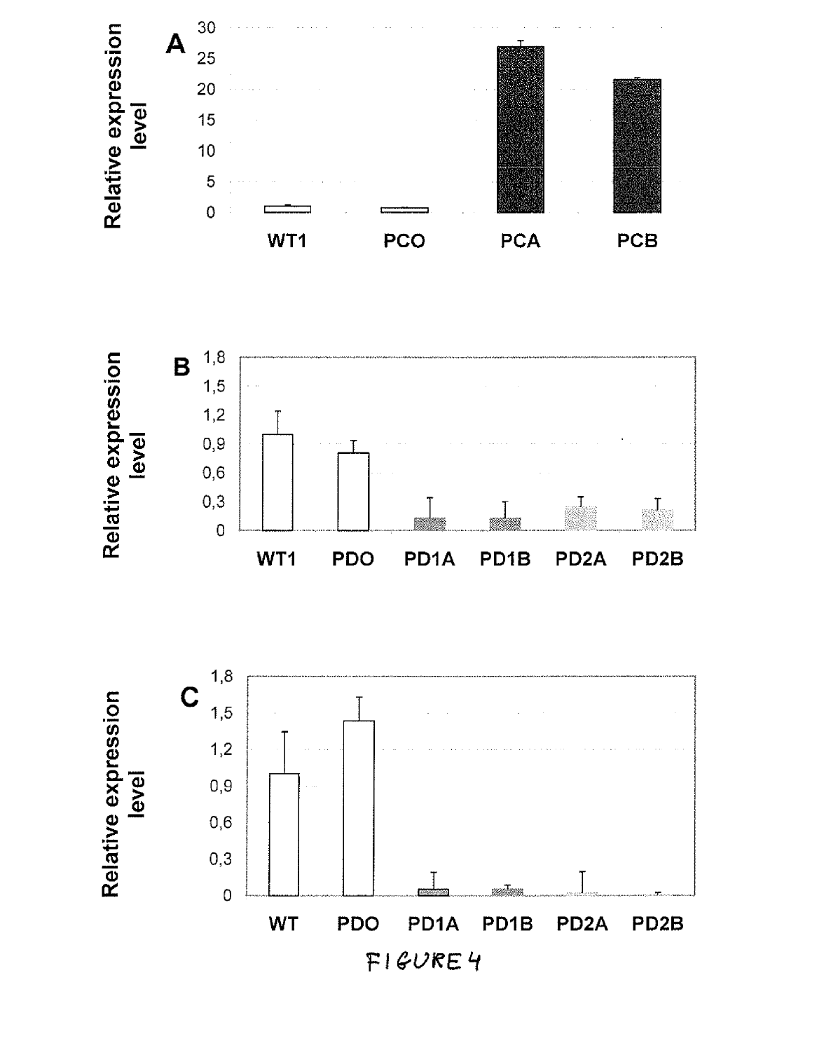

Results: the inventors have confirmed that MAD26 expression in OsMAD26 mRNA-interfered plants PD1A, PD1B, PD2A et PD2B was silenced (FIGS. 4 B and D) while the MAD26 expression level in PCA and PCB transgenic plants over-expressing the OsMAD26 is at least 20-fold more important than the MAD26 expression in control plants (FIG. 4A).

### 5. Resistance Assay Against Magnaporthe oryzae

In addition to the studied transgenic lines, O. sativa japonica cv Maratelli was used as a susceptible control. Plants were sown in trays of 40×29×7 cm filled with compost of Neuhaus S pH 4-4.5 and Pozzolana (70 liters Neuhaus S mixed with 2 shovels of Pozzolana). Ten seeds of each line were sown in rows in a tray containing 12 lines each. Plants were grown until the 4-5 leaf stage a greenhouse with a thermoperiod of 26/21° C. (day/night), a 12-h photoperiod under a light intensity of 400-600 W/m². Watering was done every day and once a week nutritive solution composed of 1.76 g/L of Algospeed (Laboratoire Algochimie, Chateau-Renault, France) and 0.125 g/L of Ferge (FERVEQ La Rochelle, France) was supplied. The GUY 11 isolate (CIRAD collection, Montpellier, France) of M. oryzae was used for inoculation. This isolate is compatible with O. Sativa cv Nipponbare and generate partial susceptible symptoms. The fungus was cultured in Petri dishes containing 20 ml of medium composed of 20 gl/l rice seed flour prepared grounding paddy rice at machine (Commerciel Blendor American) for 3 nm, 2.5 g/l yeast extract (Roth-2363.3), 1.5% agar (VWP, 20768.292) supplemented after autoclaving with 500 000 units/L of sterile penicillin G (Sigma P3032-10MU). Fungus culture was carried out in a growth chamber with a 12-h photoperiod and a constant temperature of 25° C. for 7 days. After 7 days, conidia were harvested from plates by flooding the plate with 10 ml of sterile distilled water and filtering through two layers of gauze to remove mycelium fragment from the suspension. The concentration in conidia of the suspension was adjusted to 50000 conidia ml⁻¹ and supplemented with 0.5% (w:v) of gelatin (Merck). Inoculations were performed on 4-5 leaf stage plantlets by spraying 30 ml of the conidia suspensions on each tray. Inoculated plantlets were incubated for 16 h in a controlled climatic chamber at 25° C., 95% relative humidity and transferred back to the greenhouse. After 3 to 7 days, lesions on rice leaves were categorized in resistant or susceptible categories and counted. The data presented are representative of data obtained for three independent repetitions of the experimentation.

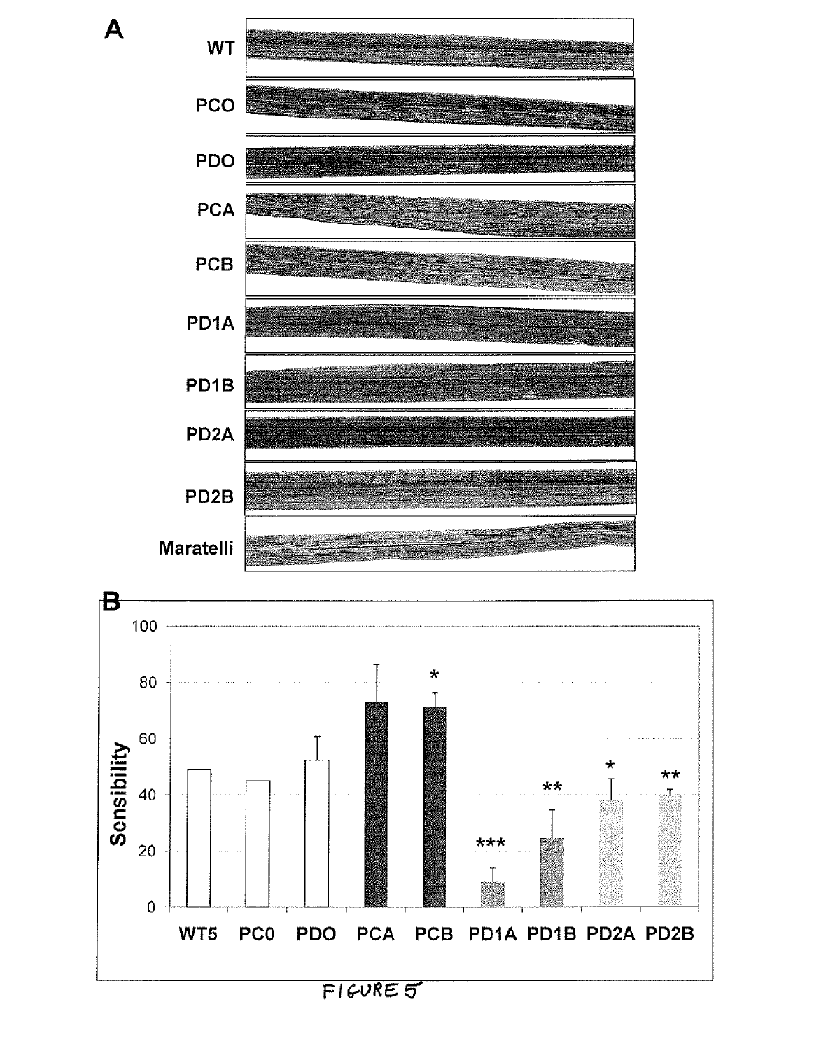

Results: the inventors have demonstrated in FIG. 5 that OsMAD26 mRNA interfered plants PD1A, PD1B, PD2A et PD2B are more resistant to fungal pathogens while PCA and PCB plants over-expressing the OsMAD26 gene are more susceptible to fungal diseases.

### 6. Resistance Assay Against Xanthomonas oryzae pv. Oryzae (Xoo)

Resistance assays against Xanthomonas oryzae pv. Oryzae (Xoo) were carried out on 2 month-old rice plants grown in the same conditions as described above for M. oryzae resistance assays. After 2 months, the plants were transferred from greenhouse to a culture chamber providing 12 h light at 28° C. (5 tubes fluorescent) and 12 h obscurity (0 tubes fluorescents) at 21° C. circadian cycles. In order to evaluate expression of genes identified as markers of defense in the different studied lines in the absence of pathogen, one month before infection, the youngest and the before youngest fully expended leaf were collected pooling 3 plantlets in the same line. This sample was used for QPCR analysis with specific primers of defense genes. The Xoo strain PXO99, a representative strain of Philippines race 6 (Song et al. 1995) was grown on PSA medium (10 gl⁻¹ peptone, 10 g/L sucrose, 1 g/L glutamic acid, 16 g/L bacto-agar, pH 7.0) for 3 days at 27° C. Bacterial blight inoculation was performed using the leaf-clipping methode described by Kauffman et al. (1973). The bacterial cells of Xoo were suspended in 50 ml sterile water to obtain an optical density of 0.5 measured at 600 nm (OD600). The bacterial cell suspension was applied to the two youngest fully expanded leaves on the main tiller of 2 months old rice plants by cutting the leaf 5-6 cm from the tip using a pair of scissors dipped in the Xoo solution. Lesion length (LL) was measured 14 days post-inoculation (dpi) according to the criteria described previously (Amante-Bordeos et al. 1992). The data presented are representative of data obtained for two independent repetitions of the experimentation. After symptom measurement, infected leaves were also collected in liquid nitrogen and used for RNA extraction and QPCR analysis to measure the expression level of different defense genes.

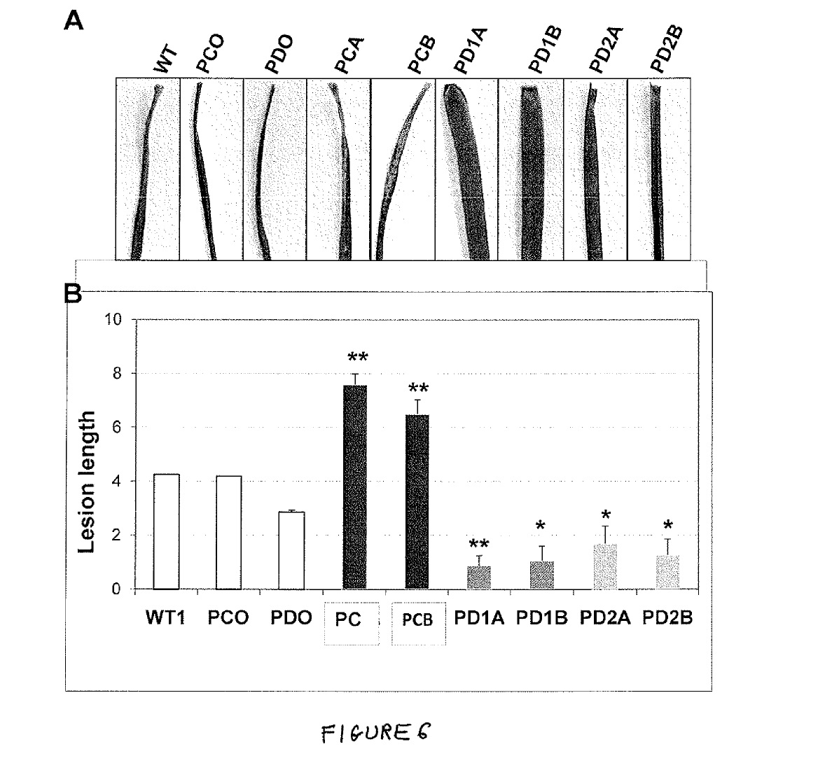

Results: the inventors have demonstrated in FIG. 6 that OsMAD26 mRNA interfered plants PD1A, PD1B, PD2A et PD2B are more resistant to bacterial pathogens while PCA and PCB plants over-expressing the OsMAD26 gene are more susceptible to bacterial diseases. Indeed, PCA and PCB plants have much more lesions than PD1A, PD1B, PD2A et PD2B plants.

### 7. Resistance Assay Against Water Stress

Plants were germinated in a one-half-strength MS liquid medium in a growth chamber for 7 d and transplanted into soil and grown in the green house at the same conditions described above. Each pot was filled with the same amount of soils (Tref, EGO 140), planted with 5 seedlings and watered with the same volume of water. After one month, plants were subjected to 18 days of withholding water followed by 15 days of watering. Drought tolerance was evaluated by determining the percentage of plants that survived or continued to grow after the period of recovery. Fv/Fm values of plants were measured each day after withholding watering with a pulse modulated fluorometer (Handy PEA, EUROSEP Instruments) as previously described (Jang et al. 2003; Oh et al. 2005). This experiment was done on 20 plants per line and repeated three times. Statistical analysis of the data obtained in these experiments was performed using the R software at a 5% confidence level. During water stress, the relative water content (RWC), of leaves was measured according to Bans and Weatherly, 1962. A mid-leaf section of about 1×7 cm was cut with scissors from the top of the most expanded leaf of five plants. The other leaves were also harvested, frozen in liquid nitrogen and stocked at −80° C. for RNA extraction and RT-qPCR analysis of the expression of stress related genes. For RWC measurement, each leaf section was pre-weighed airtight to obtain leaf sample weight (W). After that, the sample was immediately hydrated to full turgidity. The basal part of the leaf was placed to the bottom of a caped 50 ml Stardet tube containing 15 ml of de-ionized water and incubated at room temperature. After 4 h, the leaf was removed and dried quickly and lightly with filter paper and immediately weighed to obtain fully turgid weight (TW). Sample were then dried at 80° C. for 24 h and weighed to determine dry weight (DW). The RWC was calculated as following: RWC (%)=[(W−DW)/(TW−DW)]×100. Basis on the results of this calculation, the samples stocked at −80° C. of two plants were taken out. RNA extraction and RT-qPCR were performed from two plants of each line that had the same RWC, as described earlier with specific primers of genes identified as drought and high salinity stresses markers in rice: rab21, a rice dehydrin (accession number AK109096), salT (salt-stress-induced protein, accession number AF001395), and dip1 (dehydration-stress inducible protein 1, accession number AY587109) genes (Claes et al. 1990; Oh et al. 2005; Rabbani et al. 2003).

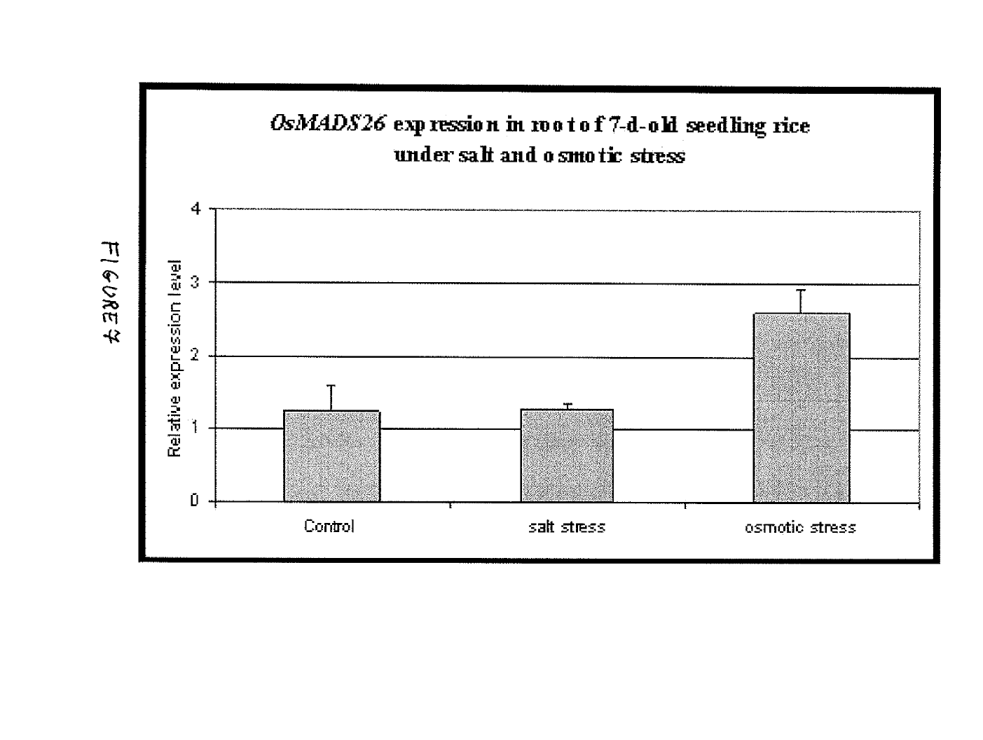

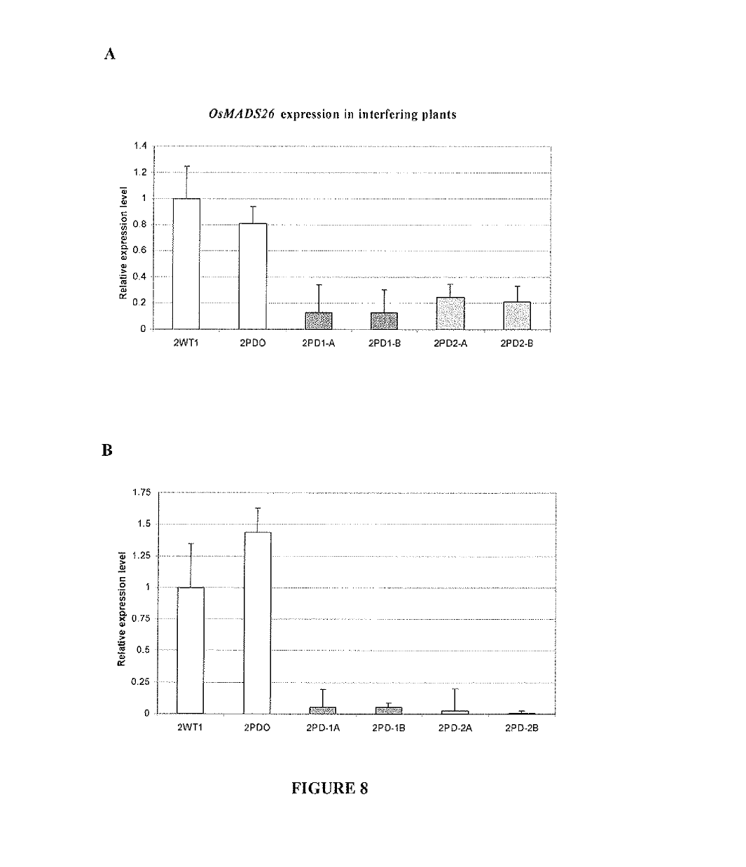

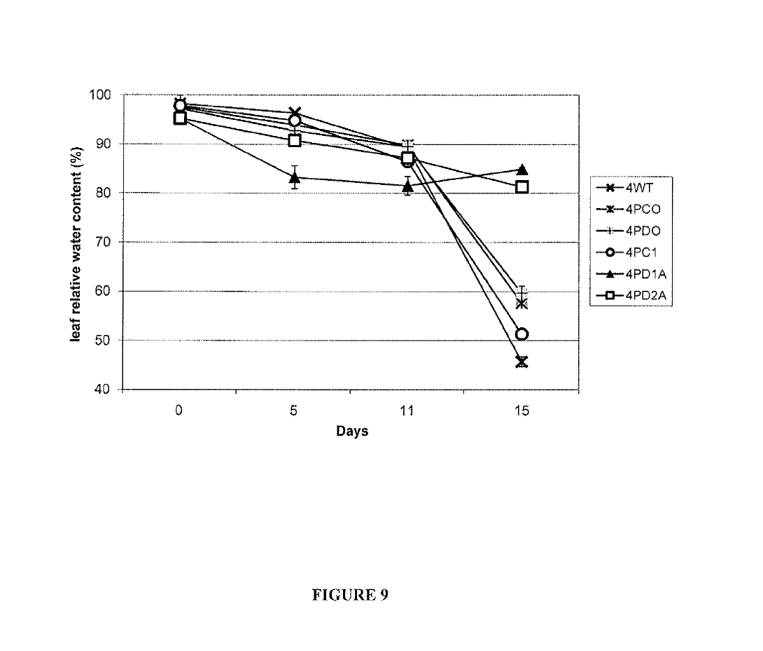

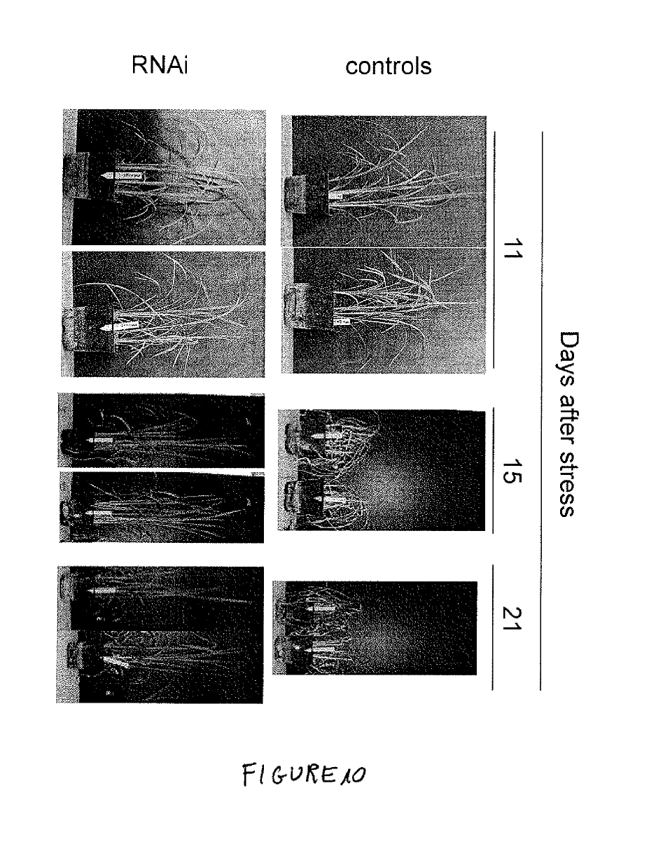

Results: the inventors have discovered that OsMAD26 gene is induced under osmotic stress (FIG. 7) and that the OsMAD26 expression profile is different in various plant organs (FIG. 1). The inventors have also demonstrated that OsMAD26 gene is silenced in RNAi-interfered plants (lines 2PD1-A, 2PD1-B, 2PD2-A, 2PD2-B) (FIG. 8A) and that under osmotic stress, the MAD26 gene is still silenced (FIG. 8B). Finally, in FIGS. 9 and 10, the inventors have demonstrated that MAD26 RNA-interfered plants are more resistant to drought stress and plants overexpressing the MAD26 gene are less resistant to drought stress.

### 8. MAD26 Orthologs

Furthermore, the inventors have carried out Tblastn searches with the MAD26 protein from rice and have identified by blastp search several putative orthologs in wheat, sorghum and maize. To see if homology uncovers phylogenetic relationship and possibly functional homology, the inventors have tested whether the cereal homologs were in turn the best blast hit (Best Blast Mutual Hit=BBMH) on rice.

TABLE 2
Orthologs of MAD26

| | SEQ ID NO MADS26 | Best homolog | % Amino |
|---|---|---|---|
| Species | best homolog | Accession (1) | acid identity |
| wheat | SEQ ID NO: 3 | CAM59056 | 65% |
| sorghum | SEQ ID NO: 5 | XP_002443744.1 | 66% |
| maize | SEQ ID NO: 7 | ABW84393 | 85% |

(1) The MADS26 protein SEQ ID NO: 2 was searched against wheat, sorghum and maize sequences using blastp in the ncbi sequence database.

### CONCLUSIONS

Altogether, the expression data and the phenotypical data indicate that the MAD26 gene is a negative regulator of resistance to Magnaporthe oryzae, to Xanthomonas oryzae and to drought stress. This is the first example ever found of a plant transcription factor of the MADS-box family negatively regulating biotic and abiotic stress response.

### Sequence Listing

SEQ ID NO: 1
>Os08g02070.1 CDS
ATGGCGCGAGGCAAGGTGCAGCTCCGTCGCATCGAGAACCCGGTTCACCGTCAGGTCACC
TTCTGCAAGCGCCGTGCCGGCCTGCTGAAGAAGGCCAGGGAGCTCTCCATCCTCTGCGAG
GCCGACATCGGCATCATCATCTTCTCCGCCCACGGCAAGCTCTACGACCTCGCCACCACC
GGAACCATGGAGGAGCTGATCGAGAGGTACAAGAGTGCTAGTGGCGAACAGGCCAACGCC
TGCGGCGACCAGAGAATGGACCCAAAACAGGAGGCAATGGTGCTCAAACAAGAAATCAAT
CTACTGCAGAAGGGCCTGAGGTACATCTATGGGAACAGGGCAAATGAACACATGACTGTT
GAAGAGCTGAATGCCCTAGAGAGGTACTTAGAGATATGGATGTACAACATTCGCTCCGCA
AAGATGCAGATAATGATCCAAGAGATCCAAGCACTAAAGAGCAAGGAAGGCATGTTGAAA
GCTGCTAACGAAATTCTCCAAGAAAAGATAGTAGAACAGAATGGTCTGATCGACGTAGGC
ATGATGGTAGCAGATCAACAGAATGGGCATTTTAGTACAGTCCCACTGTTAGAAGAGATC
ACTAACCCACTGACTATACTGAGTGGCTATTCTACTTGTAGGGGCTCGGAGATGGGCTAT
TCCTTCTAA
SEQ ID NO: 2
>Os08g02070.1 PROT
MARGKVQLRRIENPVHRQVTFCKRRAGLLKKARELSILCEADIGIIIFSAHGKLYDLATT
GTMEELIERYKSASGEQANACGDQRMDPKQEAMVLKQEINLLQKGLRYIYGNRANEHMGT
MEELIERYKSASGEQANACGDQRMDPKQEAMVLKQEINLLQKGLRYIYGNRANEHMTVEE
LNALERYLEIWMYNIRSAKMQIMIQEIQALKSKEGMLKAANEILQEKIVEQNGLIDVGMM
VADQQNGHFSTVPLLEEITNPLTILSGYSTCRGSEMGYSF*
SEQ ID NO: 3
Putative TaMADS26
>CAM59056
MARGKVQLRR IENPVHRQVT FCKRRAGLLK KARELSVLCD ADIGIIIFSA HGKLYDLATT
GTMDGLIERY KSASGEGMTG DGCGDQRVDP KQEAMVLKQE IDLLQKGLRY IYGNRANEHM
NVDELNALER YLEIWMFNIR SAKMQIMIQE IQALKSKEGM LKAANEILQE KIVEQHGLID
VGMTIADQQN GHFSTVPMLE EITNPLTILS GYSTCRGSEM GYSF
The amino acid sequence of SEQ ID NO: 4 derives from
SEQ ID NO: 4
> AM502878
atggcgagag gcaaggtcca gctccggcgc atcgagaacc ccgtccaccg gcaggtcacc
ttctgcaagc gccgcgcagg gctcctcaag aaggccaggg agctctctgt cctctgcgac
gccgacatcg gcatcatcat cttctccgca cacggcaagc tctacgacct cgccaccacc
ggaaccatgg atgggctgat cgagaggtac aagagtgcca gtggagaagg catgaccggc
gacggctgcg gcgaccagag agtggaccca aagcaggagg caatggtgct gaaacaagaa
atagaccttc tgcagaaggg actgaggtac atttatggaa acagggcaaa tgagcacatg
aatgttgacg agctgaatgc cctggagagg tacttggaga tatggatgtt caacatccgc
tccgcaaaga tgcagataat gattcaagag atccaggcac tgaagagcaa ggagggcatg
ttgaaagctg ccaacgaaat tctccaggaa aagatagtag aacagcatgg actgatcgac
gtaggcatga ctatagcaga tcagcagaat gggcatttta gtacagtccc aatgttagag
gagatcacta acccactgac tatactgagt ggctattcta cttgtagggg ctcagagatg
ggctattcct tctga
SEQ ID NO: 5
Putative sorghum MADS26
>XP_002443744.1
MARGKVQLRR IENPVHRQVT FCKRRAGLLK KARELSVLCD AHIGIIIFSA HGKLYDLATT
GTMEELIDRY KTASGEAADG SGDNRMDPKQ ETMVLQQEIN LLQKGLRYIY GNRANEHMNV
DELNALERYL EIWMYNIRSA KMQIMIQEIQ ALKSKEGMLK AANEILREKI VEQSSLLDVG
MVVADQQNGH FSTVPLIEEI TNPLTILSGY SNCRGSEMGY SF
The amino acid sequence of SEQ ID NO: 6 derives from
SEQ ID NO: 6
> XM_002443699
atggcgcggg gcaaagtgca gctgcggcgc atcgagaacc cggtgcaccg gcaggtgacc
ttctgcaagc gccgcgcggg gctgctcaag aaggcacggg agctctccgt cctctgcgac
gcccacatcg gcatcatcat cttctccgcg cacggcaagc tctacgacct cgccaccacc
gggaccatgg aagagctgat cgacaggtac aagactgcca gcggagaagc tgccgacggc
tccggcgaca acagaatgga tccaaaacaa gaaaccatgg tgctgcaaca ggaaatcaat
ctgctccaga aaggactcag gtacatctac gggaacaggg caaatgaaca catgaatgtt
gacgaactga atgcccttga gaggtacttg gagatatgga tgtacaacat ccgctctgca
aagatgcaga taatgattca agagatacaa gcactaaaaa gcaaggaagg catgttgaaa
gctgctaacg aaattctccg ggaaaagata gtagaacaga gtagtttgct tgatgtaggc
atggtggtag cggatcaaca gaatgggcat tttagtacag tcccactgat agaagagatc
actaacccac tgactatact gagtggatat tctaactgta ggggctcaga gatgggctat
tccttctaa
SEQ ID NO: 7
Putative Zea mays MADS26
>ABW84393
MGRGKVQLKR IENKINRQVT FSKRRSGLLK KAHEISVLCD AEVALIIFST KGKLYEYSTD
SCMDKILDRY ERYSYAEKVL ISVESETQGN WCHEYRKLKA KVETIQKCQK HLMGEDLETL
NLKELQQLEQ QLESSLKHIR TRKSQLMLES ISELQRKEKS LQEENKVLQK ELAEKQKAQR
KQVQWGQTQQ QTSSSSSCFM IREAAPTTNI SIFPVAAGGR LVEGAAAQPQ ARVGLPPWML
SHLSS
The amino acid sequence of SEQ ID NO: 8 derives from
SEQ ID NO: 8
> EU012444
atggggcgcg gtaaggtgca gctgaagcgg atcgagaaca agatcaaccg ccaggtgacc
ttctccaagc gccgctcggg gctgctcaag aaggcgcacg agatctccgt gctctgcgac
gccgaggtcg cgctcatcat cttctccacc aaagggaagc tctacgagta ttccaccgat
tcatgtatgg acaaaattct tgaccggtac gagcgctact cctatgcaga aaaggttctt
atttcagtag aatctgaaac tcagggcaat tggtgccacg agtatagaaa actaaaggcg
aaggtcgaga caatacaaaa atgtcaaaag cacctcatgg gagaggatct tgaaacgttg
aatctcaaag agcttcagca actagagcag cagctggaga gttcactgaa acatatcaga
accaggaaga gccagcttat gctcgagtca atttcggagc tccaacggaa ggagaagtcg
ctgcaggagg agaacaaggt tctgcagaag gagctcgcgg agaagcagaa agcccagcgg
aagcaagtgc aatggggcca aacccaacag cagaccagtt cgtcttcctc gtgcttcatg
ataagggaag ctgccccaac aacaaatatc agcatttttc ctgtggcagc aggcgggagg
ttggtggaag gtgcagcagc gcagccacag gctcgcgttg gactaccacc atggatgctt
agccacctga gcagctga
SEQ ID NO: 9
MAD33
>Os12g10520.1
MVRGKVQMRRIENPVHRQVTFCKRRGGLLKKARELSVLCDADVGVIIFSSQGKLHELATN
GNMHNLVERYQSNVAGGQMEPGALQRQQVAEQGIFLLREEIDLLQRGLRSTYGGGAGEMT
LDKLHALEKGLELWIYQIRTIKMQMMQQEIQFLRNKEGILKEANEMLQEKVKEQQKLYMS
LLDLHSQQPIQPMTYGNRFFSI*
SEQ ID NO: 10
MAD14
>Os03g54160.1
MGRGKVQLKRIENKINRQVIFSKRRSGLLKKANEISVLCDAEVALIIFSTKGKLYEYATD
SCMDKILERYERYSYAEKVLISAESDIQGNWCHEYRKLKAKVETIQKCQKHLMGEDLESL
NLKELQQLEQQLENSLKHIRSRKSQLMLESINELQRKEKSLQEENKVLQKENPCSFLQLV
EKQKVQKQQVQWDQTQPQTSSSSSSFMMREALPTINISNYPAAAGERIEDVAAGQPQHVR
IGLPPWMLSHING*
SEQ ID NO: 11
Putative HvMAD26
>CAB97351
MGRGPVQLRR IENKINRQVT FSKRRSGLLK KAHEISVLCD AEVALIVFST KGKLYEYSSQ
DSSMDVILER YQRYSFEERA VLDPSTGDQA NWGDEYGSLK IKLDALQKSQ RQLLGEQLDP
LTTKELQQLE QQLDSSLKHI RSRKNQLLFE SISELQKKEK SLKDQNGVLQ KHLVETEKEK
NNVLSNIHHR EQLNEATNIH HQEQLSGATT SSPSPTPPTA QDSMAPPNIG PYQSRGGGDP
EPQPSPAQAN NSNLPPWMLR TIGNR
SEQ ID NO: 12
Putative HvMAD26
> CAB97355
MGRGRVELKR IENKINRQVT FAKRRNGLLK KAYELSVLCD AEVALIVFSN RGKLYEFCST
QSMTKTLDKY QKCSYAGPET TVQNRENEQL KNSRNEYLKL KTRVDNLQRT QRNLLGEDLD
SLGIKELESL EKQLDSSLKH IRTTRTQHMV DQLTELQRRE QMFSEANKCL RIKLEESNQV
HGQQLWEHNN NVLSYERQPE VQPQMHGGNG FFHPLDAAGE PTLHIGYPPE SLNSSCMTTF
MPPWLP
SEQ ID NO: 13
Putative HvMAD26
> AAW82994
MGRGKVQLKR IENKINRQVT FSKRRSGLLK KAHEISVLCD AEVGLIIFST KGKLYEFSTE
SCMDKILERY ERYSYAEKVL VSSESEIQGN WCHEYRKLKA KVETIQKCQK HLMGEDLESL
NLKELQQLEQ QLESSLKHIR ARKNQLMHES ISELQKKERS LQEENKVLQK ELVEKQKAQA
AQQDQTQPQT SSSSSSFMMR DAPPVADTSN HPAAAGERAE DVAVQPQVPL RTALPLWMVS
HING
SEQ ID NO: 14
Putative HvMAD26
> CAB97354
MGRGKVQLKR IENKINRQVT FSKRRNGLLK KAHEISVLCD AEVAVIVFSP KGKLYEYATD
SSMDKILERY ERYSYAEKAL ISAESESEGN WCHEYRKLKA KIETIQKCHK HLMGEDLDSL
NLKELQQLEQ QLESSLKHIR SRKSHLMMES ISELQKKERS LQEENKALQK ELVERQKAAS
RQQQLQQQQQ QQQMQWEHQA QTQTHTHTQN QPQAQTSSSS SSFMMRDQQA HAPQQNICSY
PPVTMGGEAT AAAAAPEQQA QLRICLPPWM LSHLNA
SEQ ID NO: 15
Putative HvMAD26
> ACB4530
MGRGRVELKR IENKINRQVT FAKRRNGLLK KAYELSVLCD AEVALIIFSN RGKLYEFCSG
QSMPKTLERY QKCSYGGPDT AIQNKENELV QSSRNEYLKL KARVENLQRT QRNLLGEDLG
SLGIKDLEQL EKQLDSSLRH IRSTRTQHML DQLTDLQRKE QMLSEANKCL RRKLEESSQQ
MQGQMWEQHA ANLLGYDHLR QSPHQQQAQH HGGNGFFHPL DPTTEPTLQI GYTQEQINNA
CVAASFMPTW LP
SEQ ID NO: 16
GST1 (215 bp) specific of OsMAD26 (see FIG. 3)
SEQ ID NO: 17
GST2 (321 bp) specific of OsMAD26 (see FIG. 3)

### REFERENCES

Alvarez-Buylla, E. R., Liljegren, S. J., Pelaz, S., Gold, S. E., Burgeff, C., Ditta, G. S., Vergara-Silva, F., and Yanofsky, M. F. (2000). MADS-box gene evolution beyond flowers: expression in pollen, endosperm, guard cells, roots and trichomes. Plant J 24, 457-466.
Arora, R., Agarwal, P., Ray, S., Singh, A. K., Singh, V. P., Tyagi, A. K., and Kapoor, S. (2007). MADS-box gene family in rice: genome-wide identification, organization and expression profiling during reproductive development and stress. BMC Genomics 8, 242.
Fang, S. C., and Fernandez, D. E. (2002). Effect of regulated overexpression of the MADS domain factor AGL15 on flower senescence and fruit maturation. Plant Physiol 130, 78-89.
Fernandez, D. E., Heck, G. R., Perry, S. E., Patterson, S. E., Bleecker, A. B., and Fang, S. C. (2000). The embryo MADS domain factor AGL15 acts postembryonically. Inhibition of perianth senescence and abscission via constitutive expression. Plant Cell 12, 183-198.
Khush, G. (2005) What it will take to Feed 5.0 Billion Rice consumers in 2030. Plant Molecular Biology, 59(1): 1-6.
Khush, G S. and Jena, K K., (2009) Current status and future prospects for research on blast resistance in rice (Oryza sativa L.) In G L Wang (ed.) and B Valent (ed.). Advances in Genetics, Genomics and Control of Rice Blast Disease, p. 1-10, Springer.
Lee, S., Woo, Y. M., Ryu, S. I., Shin, Y. D., Kim, W. T., Park, K. Y., Lee, I. J., and An, G. (2008). Further characterization of a rice AGL12 group MADS-box gene, OsMAD26. Plant Physiol 147, 156-168.
Liljegren, S. J., Ditta, G. S., Eshed, Y., Savidge, B., Bowman, J. L., and Yanofsky, M. F. (2000). SHATTERPROOF MADS-box genes control seed dispersal in Arabidopsis. Nature 404, 766-770.
Mao, L., Begum, D., Chuang, H. W., Budiman, M. A., Szymkowiak, E. J., Irish, E. E., and Wing, R. A. (2000). JOINTLESS is a MADS-box gene controlling tomato flower abscission zone development. Nature 406, 910-913.
Messenguy, F., and Dubois, E. (2003). Role of MADS box proteins and their cofactors in combinatorial control of gene expression and cell development. Gene 316, 1-21.
Montiel, G., Breton, C., Thiersault, M., Burlat, V., Jay-Allemand, C., and Gantet, P. (2007). Transcription factor Agamous-like 12 from Arabidopsis promotes tissue-like organization and alkaloid biosynthesis in Catharanthus roseus suspension cells. Metab Eng 9, 125-132.
Parenicova, L., de Folter, S., Kieffer, M., Horner, D. S., Favalli, C., Busscher, J., Cook, H. E., Ingram, R. M., Kater, M. M., Davies, B., Angenent, G. C., and Colombo, L. (2003). Molecular and phylogenetic analyses of the complete MADS-box transcription factor family in Arabidopsis: new openings to the MADS world. Plant Cell 15, 1538-1551.
Shore, P., and Sharrocks, A. D. (1995). The MADS-box family of transcription factors. Eur J Biochem 229, 1-13.
Tapia-Lopez, R., Garcia-Ponce, B., Dubrovsky, J. G., Garay-Arroyo, A., Perez-Ruiz, R. V., Kim, S. H., Acevedo, F., Pelaz, S., and Alvarez-Buylla, E. R. (2008). An AGAMOUS-related MADS-box gene, XAL1 (AGL12), regulates root meristem cell proliferation and flowering transition in Arabidopsis. Plant Physiol 146, 1182-1192.
Theissen, G., Becker, A., Di Rosa, A., Kanno, A., Kim, J. T., Munster, T., Winter, K. U., and Saedler, H. (2000). A short history of MADS-box genes in plants. Plant Mol Biol 42, 115-149.
Vrebalov, J., Ruezinsky, D., Padmanabhan, V., White, R., Medrano, D., Drake, R., Schuch, W., and Giovannoni, J. (2002). A MADS-box gene necessary for fruit ripening at the tomato ripening-inhibitor (rin) locus. Science 296, 343-346.

1. A monocot plant having a defective MADS26 gene function and exhibiting an increased resistance to biotic and/or abiotic stress.

2. (canceled)

3. The plant of claim 1, wherein said MADS26 gene function is defective as a result of a deletion, insertion and/or substitution of one or more nucleotides, site-specific mutagenesis, ethyl methanesulfonate (EMS) mutagenesis, targeting induced local lesions in genomes (TILLING), knock-out techniques, or by gene silencing induced by RNA interference.

4. (canceled)

5. The plant of claim 1, wherein said monocot plant is of the Poaceae family.

6. The plant of claim 5, wherein said plant is a cereal selected from rice, wheat, barley, oat, rye, sorghum or maize.

7. A seed of the plant of claim 6.

8. (canceled)

9. The plant of claim 1, wherein said resistance to biotic stress is a resistance to fungal and/or bacterial pathogens.

10. The plant of claim 9, wherein said fungal pathogens are selected from Magnaporthe, Puccinia, Ustilago, Septoria, Erisyphe, Rhizoctonia and or Fusarium species.

11. The plant of claim 10, wherein said fungal pathogen is Magnaporthe oryzae.

12. The plant of claim 9, wherein said bacterial pathogens are selected from Xanthomonas, Ralstonia, Erwinia, Pectobacterium, Pantoea, Agrobacterium, Pseudomonas, Burkholderia, Acidovorax, Clavibacter, Streptomyces, Xylella, Spiroplasma and Phytoplasma species.

13. The plant of claim 12, wherein said bacterial pathogen is Xanthomonas oryzae.

14. The plant of claim 1, wherein said resistance to abiotic stress is a resistance to drought stress.

15. (canceled)

16. A method for producing a monocot plant having increased resistance to fungal and/or bacterial pathogens or to drought stress, wherein the method comprises:
(a) inactivation of a MADS26 gene function in a plant cell;
(b) optionally, selection of plant cells of step (a) with inactivated MADS26 gene function;
(c) regeneration of plants from cells of step (a) or (b); and
(d) optionally, selection of a plant of (c) with increased resistance to fungal and/or bacterial pathogens or to drought stress, said plant having a defective MADS26 gene function.

17. The method according to claim 16, wherein said MADS26 gene function is inactivated by deletion, insertion and/or substitution of one or more nucleotides, site-specific mutagenesis, ethyl methanesulfonate (EMS) mutagenesis, targeting induced local lesions in genomes (TILLING), knock-out techniques, or by gene silencing induced by RNA interference.

18. The method according to claim 16, wherein the plant is a monocot selected from the Poaceae family.

19. (canceled)

20. An RNAi molecule that inhibits the expression of the MAD26 gene.

21. The RNAi molecule of claim 20 that binds to MAD26 mRNA sequence which is complementary to a sequence comprising the sequence of SEQ ID NO: 16 (GST1) or SEQ ID NO: 17 (GST2).

22. A method for increasing resistance of monocot plants or plant cells thereof to biotic or abiotic stress which comprises inactivating a MADS-box gene of said plant or plant cells.

23-27. (canceled)

28. A plant transformed with a vector comprising a nucleic acid sequence expressing an RNAi molecule that inhibits the expression of a MADS26 gene.

29. The plant of claim 28, wherein the nucleic acid sequence comprises the sequence of SEQ ID NO: 16 or 17.

30. A method of claim 16, comprising:
(a) inactivation of MADS26 gene function in seeds by mutagenesis;
(b) generation of plants from the seeds of step (a); and
(c) selection of a plants of (b) having a defective MADS26 gene function.
